(19) United States
(12) Patent Application Publication
(10) Pub. No.: US 2025/0072834 A1
(43) Pub. Date: Mar. 6, 2025

(54) PROVIDING USER PROMPTS CORRESPONDING TO WEARABLE DEVICE SENSOR POSITIONS

(71) Applicant: QUALCOMM Incorporated, San Diego, CA (US)

(72) Inventors: Emily Kathryn BROOKS, Amherst, NY (US); John Keith SCHNEIDER, Williamsville, NY (US); Nicholas BUCHAN, San Jose, CA (US); Evan Michael BRELOFF, Kenmore, NY (US); Ye ZHAN, Buffalo, NY (US); Vivekananda Parampalli ADIGA, Williamsville, NY (US); Shounak Uday GORE, Hyderabad (IN)

(21) Appl. No.: 18/461,416

(22) Filed: Sep. 5, 2023

(51) Int. Cl. A61B 5/00; A61B 5/024

(52) U.S. Cl. CPC A61B 5/684; A61B 5/02427; A61B 5/6802; A61B 5/6843; A61B 5/7203; A61B 5/7246; A61B 5/74

(57) ABSTRACT

Some disclosed examples involve obtaining, via a sensor system of a wearable device, first heart rate waveforms at a first wearable device configuration corresponding with a first position at least a portion of the sensor system. Some examples involve prompting, via a user interface of the wearable device, a user to change the wearable device configuration to a second wearable device configuration, the second wearable device configuration corresponding with a second position of one or more sensors of the sensor system. Some examples involve obtaining second heart rate waveforms at the second wearable device configuration and determining, based at least in part on the first heart rate waveforms and the second heart rate waveforms, whether to change a wearable device configuration or maintain a current wearable device configuration. Some examples involve prompting the user either to change the wearable device configuration or maintain the current wearable device configuration.

### TECHNICAL FIELD

This disclosure relates generally to wearable devices that include sensor systems configured for biometric applications, biomedical applications, or both.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and ambulatory monitoring. Some such devices are, or include, photoacoustic devices. Although some previously-deployed photoacoustic devices and systems can provide acceptable results, improved photoacoustic devices and systems would be desirable.

### SUMMARY

The systems, methods and devices of this disclosure each have several aspects, no single one of which is solely responsible for the desirable attributes disclosed herein.

One innovative aspect of the subject matter described in this disclosure can be implemented in an apparatus. In some implementations, the apparatus may be, or may include, a wearable device. The apparatus may include a sensor system, a user interface system and a control system. The sensor system may, in some examples, be adjustable according to a plurality of configurations, which may be referred to herein as “wearable device configurations.” In some examples, the sensor system may be configured to obtain heart rate waveforms from a user of the wearable device. According to some examples, the sensor system may be, or may include, a photoacoustic sensor system. The photoacoustic sensor system may include a light source system and a receiver system. The receiver system may be, or may include, an ultrasonic receiver system having an array of ultrasonic receiver elements.

In some implementations, the apparatus may include a control system. The control system may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system may be configured to obtain, via the sensor system, first heart rate waveforms at a first wearable device configuration, the first wearable device configuration corresponding with a first position of one or more sensors of the sensor system and to control the user interface system to prompt the user to change the wearable device configuration to a second wearable device configuration, the second wearable device configuration corresponding with a second position of one or more sensors of the sensor system. The control system may be configured to obtain, via the sensor system, second heart rate waveforms at the second wearable device configuration, to determine, based at least in part on the first heart rate waveforms and the second heart rate waveforms, whether to change a wearable device configuration or maintain a current wearable device configuration; and to control the user interface system to prompt the user either to change the wearable device configuration or maintain the current wearable device configuration.

In some examples, determining whether to change a wearable device configuration or maintain a current wearable device configuration may involve determining a first signal-to-noise ratio (SNR) corresponding to the first heart rate waveforms, determining a second SNR corresponding to the second heart rate waveforms and comparing the first SNR to the second SNR. In some examples, the control system may be configured to obtain, via the sensor system, first through Nth heart rate waveforms at first through Nth wearable device configurations, N being an integer greater than 2. In some examples, the control system may be configured to select, based at least in part on the first through Nth heart rate waveforms, one of the first through Nth wearable device configurations and to control the user interface system to prompt the user to change the wearable device configuration to a selected wearable device configuration.

Other innovative aspects of the subject matter described in this disclosure can be implemented in a method. The method may involve obtaining, via a sensor system of a wearable device, first heart rate waveforms at a first wearable device configuration, the first wearable device configuration corresponding with a first position of one or more sensors of the sensor system. The method may involve prompting, via a user interface of the wearable device, a user to change the wearable device configuration to a second wearable device configuration, the second wearable device configuration corresponding with a second position of one or more sensors of the sensor system. The method may involve obtaining, via the sensor system, second heart rate waveforms at the second wearable device configuration and determining, based at least in part on the first heart rate waveforms and the second heart rate waveforms, whether to change a wearable device configuration or maintain a current wearable device configuration. The method may involve prompting, via the user interface, the user either to change the wearable device configuration or maintain the current wearable device configuration.

In some examples, determining whether to change a wearable device configuration or maintain a current wearable device configuration may involve determining a first signal-to-noise ratio (SNR) corresponding to the first heart rate waveforms, determining a second SNR corresponding to the second heart rate waveforms and comparing the first SNR to the second SNR. In some examples, the method may involve obtaining, via the sensor system, first through Nth heart rate waveforms at first through Nth wearable device configurations, N being an integer greater than 2. In some examples, the method may involve selecting, based at least in part on the first through Nth heart rate waveforms, one of the first through Nth wearable device configurations and controlling a the user interface system to prompt the user to change the wearable device configuration to a selected wearable device configuration.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon. The software may include instructions for controlling one or more devices to perform one or more disclosed methods.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 is a block diagram that shows example components of an apparatus according to some disclosed implementations.

FIG. 2A shows example components of an apparatus according to some disclosed implementations.

FIGS. 2B, 2C, 2D, 2E and 2F show examples of graphical user interfaces (GUIs) that may be presented via a display system of an apparatus according to some disclosed implementations.

FIG. 2G shows example components of an apparatus according to some disclosed implementations.

FIGS. 3A, 3B and 3C show different examples of how some components of the apparatus shown in FIG. 2G may be arranged.

FIG. 3D shows examples of the components of the apparatus shown in FIG. 2G arranged with additional components.

FIG. 4 shows another example of an ultrasonic receiver element array.

FIG. 5 shows an example of an apparatus that is configured to perform a receiver-side beamforming process.

FIG. 6 is a flow diagram that shows examples of some disclosed operations.

FIG. 7 shows examples of heart rate waveform (HRW) features that may be extracted according to some implementations.

FIG. 8 shows examples of devices that may be used in a system for estimating blood pressure based, at least in part, on pulse transit time (PTT).

FIG. 9 shows a cross-sectional side view of a diagrammatic representation of a portion of an artery 900 through which a pulse 902 is propagating.

FIG. 10A shows an example ambulatory monitoring device 1000 designed to be worn around a wrist according to some implementations.

FIG. 10B shows an example ambulatory monitoring device 1000 designed to be worn on a finger according to some implementations.

FIG. 10C shows an example ambulatory monitoring device 1000 designed to reside on an earbud according to some implementations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

Wearable and non-invasive health monitoring devices, including but not limited to photoacoustic plethysmography (PAPG)-capable devices, have various potential advantages over more invasive health monitoring devices such as cuff-based or catheter-based blood pressure measurement devices. However, it has proven to be difficult to design satisfactory PAPG-based devices. One challenge is that the signal-to-noise ratio (SNR) for signals of interest, such as signals corresponding to ultrasound caused by the photoacoustic response of arterial walls, is low. For example, the signals corresponding to arterial walls are significantly lower in amplitude than signals corresponding to the photoacoustic response of skin.

Another challenge is that the orientation of the same artery may vary from user to user and within the body of the same user. These variations in arterial orientation can make it challenging to properly position the ultrasonic receiver of a PAPG-based device, or of any wearable device that includes a sensor system configured to obtain heart rate waveforms or other signals of interest relating to blood vessels or blood within the blood vessels. Moreover, when a user of a wearable device performs physical activities, such as exercising, house work, etc., the position of the wearable device relative to the user may change. Small changes in the sensor system position may result in significant differences in the received heart rate waveform signals, such as signals corresponding to arterial walls. If the sensor system is not properly positioned, signal quality may be poor and depth discrimination may not be possible.

In some implementations, a wearable device may include a sensor system, a user interface system and a control system. The sensor system may be adjustable according to a plurality of wearable device configurations. Each wearable device configuration may correspond to a different position of at least a portion of the sensor system, such as a position of one or more sensors of the sensor system. In some examples, the sensor system may be configured to obtain heart rate waveforms from a user of the wearable device. According to some examples, the control system may be configured to obtain signals, via the sensor system-which may include signals relating to heart rate waveforms—at each wearable device configuration of a plurality of wearable device configurations. In some examples, the control system may be configured to determine, based on the heart rate waveforms, whether to change a wearable device configuration or maintain a current wearable device configuration. The determination may, for example, be based at least in part on a comparison of signal-to-noise ratios (SNRs) corresponding to the signals obtained at each wearable device configuration. According to some examples, the control system may be configured to control the user interface system to prompt the user either to change the wearable device configuration or maintain the current wearable device configuration. In some examples, the sensor system may be, or may include, a photoacoustic sensor system. The photoacoustic sensor system may include a light source system and a receiver system. The receiver system may be, or may include, an ultrasonic receiver system having an array of ultrasonic receiver elements.

Particular implementations of the subject matter described in this disclosure can be implemented to realize one or more of the following potential advantages. Various disclosed implementations provide wearable devices having a sensor system that is adjustable according to a plurality of wearable device configurations and a control system that is configured to select an optimal wearable device configuration from among the plurality of wearable device configurations. The control system may be configured to control the user interface system to prompt the user either to change the wearable device configuration or maintain the current wearable device configuration. The prompt may, for example, be based on a comparison of SNRs corresponding to signals obtained at each wearable device configuration. The prompt may, for example, correspond with the wearable device configuration corresponding with the highest SNR of one or more signals of interest, such as signals corresponding to one or more blood vessel features. Accordingly, some implementations have the potential advantage of selecting an optimal wearable device configuration for obtaining one or more signals of interest. The control system may, in some examples, be configured to estimate blood pressure based, at least in part, on the blood vessel features. Enhancing the SNR of signals corresponding to blood vessel features may result in relatively more accurate blood pressure estimations.

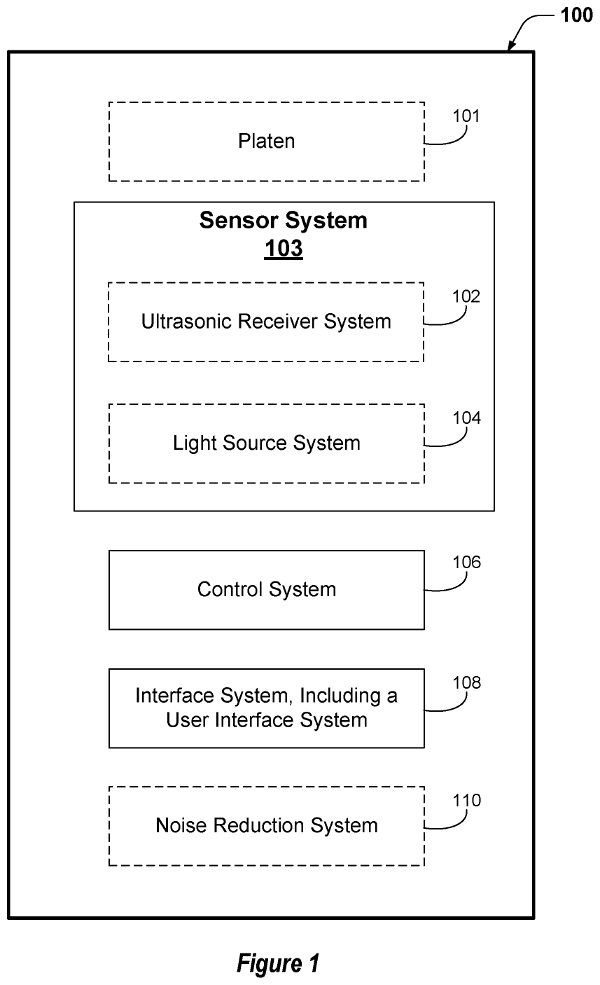

FIG. 1 is a block diagram that shows example components of an apparatus according to some disclosed implementations. In this example, the apparatus 100 includes a sensor system 103, a control system 106 and an interface system 108 that includes a user interface system. In some examples, the sensor system 103 may be, or may include, a PAPG system that includes an ultrasonic receiver system 102 and a light source system 104. According to some examples, the sensor system 103 may include one or more other types of sensors capable of detecting heart rate waveforms, such as a photoplethysmography (PPG) system, one or more microphones, one or more accelerometers, etc. Some implementations of the apparatus 100 may include a platen 101, a noise reduction system 110, or both. As with other disclosed implementations, in some alternative implementations the apparatus 100 may include more components, fewer components or different components.

According to some examples, the platen 101—when present—or another portion of the apparatus may include one or more anti-reflective layers. In some examples, one or more anti-reflective layers may reside on, or proximate, one or more outer surfaces of the platen 101.

In some examples, at least a portion of the outer surface of the platen 101—when present—may have an acoustic impedance that is configured to approximate an acoustic impedance of human skin. The portion of the outer surface of the platen 101 may, for example, be a portion that is configured to receive a target object, such as a human digit. (As used herein, the terms “finger” and “digit” may be used interchangeably, such that a thumb is one example of a finger.) A typical range of acoustic impedances for human skin is 1.53-1.680 MRayls. In some examples, at least an outer surface of the platen 101 may have an acoustic impedance that is in the range of 1.4-1.8 MRayls, or in the range of 1.5-1.7 MRayls.

Alternatively, or additionally, in some examples at least an outer surface of the platen 101 may be configured to conform to a surface of human skin. In some such examples, at least an outer surface of the platen 101 may have material properties like those of putty or chewing gum.

In some examples, at least a portion of the platen 101 may have an acoustic impedance that is configured to approximate an acoustic impedance of one or more receiver elements of the ultrasonic receiver system 102. According to some examples, a layer residing between the platen 101 and one or more receiver elements may have an acoustic impedance that is configured to approximate an acoustic impedance of the one or more receiver elements. Alternatively, or additionally, in some examples a layer residing between the platen 101 and one or more receiver elements may have an acoustic impedance that is in an acoustic impedance range between an acoustic impedance of the platen and an acoustic impedance of the one or more receiver elements.

Various examples and configurations of ultrasonic receiver systems 102 are disclosed herein. Some examples are described in more detail below. In some examples, the ultrasonic receiver system 102—when present—may include a piezoelectric receiver layer, such as a layer of PVDF polymer, a layer of PVDF-TrFE copolymer, or a layer of piezoelectric composite material. In some implementations, other piezoelectric materials may be used in the piezoelectric layer, such as aluminum nitride (AlN) or lead zirconate titanate (PZT).

In some examples, the ultrasonic receiver system 102 includes an array of ultrasonic receiver elements. In some examples, the ultrasonic receiver system 102 may include an array of electrodes arranged on a piezoelectric receiver layer, such as a layer of PVDF polymer, PVDF-TrFE copolymer, AlN or PZT, or on a layer of piezoelectric composite material. The ultrasonic receiver system 102 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the ultrasonic receiver system 102 may be, or may include, an ultrasonic receiver array. In some examples, the apparatus 100 may include one or more separate ultrasonic transmitter elements. In some such examples, the ultrasonic transmitter(s) may include an ultrasonic plane-wave generator. According to some examples, the ultrasonic receiver system 102 may include an array of ultrasonic receiver elements residing in a receiver plane.

According to some implementations, the light source system 104—when present—may include one or more light-emitting diodes (LEDs). In some implementations, the light source system 104 may include one or more laser diodes. According to some implementations, the light source system 104 may include one or more vertical-cavity surface-emitting lasers (VCSELs). In some implementations, the light source system 104 may include one or more edge-emitting lasers. In some implementations, the light source system may include one or more neodymium-doped yttrium aluminum garnet (Nd:YAG) lasers. The light source system 104 may, in some examples, include an array of light-emitting elements, such as an array of LEDs, an array of laser diodes, an array of VCSELs, an array of edge-emitting lasers, or combinations thereof.

According to some examples, the light source system 104 may include one or more light-directing elements configured to direct light from the light source system towards the target object along the first axis. In some examples, the one or more light-directing elements may include at least one diffraction grating. Alternatively, or additionally, the one or more light-directing elements may include at least one lens.

The light source system 104 may, in some examples, be configured to transmit light in one or more wavelength ranges. In some examples, the light source system 104 may configured for transmitting light in a wavelength range of 500 to 600 nanometers. According to some examples, the light source system 104 may configured for transmitting light in a wavelength range of 800 to 950 nanometers.

The light source system 104 may include various types of drive circuitry, depending on the particular implementation. In some disclosed implementations, the light source system 104 may include at least one multi-junction laser diode, which may produce less noise than single-junction laser diodes. In some examples, the light source system 104 may include a drive circuit (also referred to herein as drive circuitry) configured to cause the light source system to emit pulses of light at pulse widths in a range from 3 nanoseconds to 1000 nanoseconds. According to some examples, the light source system 104 may include a drive circuit configured to cause the light source system to emit pulses of light at pulse repetition frequencies in a range from 1 kilohertz to 100 kilohertz.

In some examples, the light source system 104 may include a light source system surface having a normal that is parallel, or substantially parallel, to the first axis. In some such examples, a light source of the light source system may reside on, or proximate, the light source system surface.

In some implementations, the light source system 104 may be configured for emitting various wavelengths of light, which may be selectable to trigger acoustic wave emissions primarily from a particular type of material. For example, because the hemoglobin in blood absorbs near-infrared light very strongly, in some implementations the light source system 104 may be configured for emitting one or more wavelengths of light in the near-infrared range, in order to trigger acoustic wave emissions from hemoglobin. However, in some examples the control system 106 may control the wavelength(s) of light emitted by the light source system 104 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the ultrasonic receiver system 102. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying RGDs) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 104 may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system 104 may be configured for emitting a plurality of light pulses at a pulse repetition frequency between about 1 MHz and about 100 MHZ. Alternatively, or additionally, in some implementations the light source system 104 may be configured for emitting a plurality of light pulses at a pulse repetition frequency between about 10 Hz and about 1 MHZ. In some examples, the pulse repetition frequency of the light pulses may correspond to an acoustic resonant frequency of the ultrasonic receiver and the substrate. For example, a set of four or more light pulses may be emitted from the light source system 104 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 104. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

The control system 106 may include one or more general purpose single- or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 106 also may include (and/or be configured for communication with) one or more memory devices, such as one or more random access memory (RAM) devices, read-only memory (ROM) devices, etc. Accordingly, the apparatus 100 may have a memory system that includes one or more memory devices, though the memory system is not shown in FIG. 1. The control system 106 may be configured for receiving and processing data from the ultrasonic receiver system 102, e.g., as described below. If the apparatus 100 includes an ultrasonic transmitter, the control system 106 may be configured for controlling the ultrasonic transmitter. In some implementations, functionality of the control system 106 may be partitioned between one or more controllers or processors, such as a dedicated sensor controller and an applications processor of a mobile device.

In some examples, the control system 106 may be configured to obtain, via the sensor system 103, first heart rate waveforms at a first wearable device configuration. The first wearable device configuration may correspond with a first position of one or more sensors of the sensor system 103. For example, in some implementations in which the sensor system 103 includes an ultrasonic receiver system 102, the first wearable device configuration may correspond with a first position of at least a portion of the ultrasonic receiver system 102. In some implementations in which the sensor system 103 includes a light source system 104, the first wearable device configuration may correspond with a first position of at least a portion of the light source system 104.

According to some examples, the control system 106 may be configured to control the user interface system to prompt a user to change the wearable device configuration to a second wearable device configuration. According to some such examples, the control system 106 may be configured to control at least one display of the user interface system, at least one loudspeaker of the user interface system, or both, to provide the user prompt. In this example, the second wearable device configuration corresponds with a second position of one or more components of the sensor system 103. In some examples, the control system 106 may be configured to obtain, via the sensor system 103, second heart rate waveforms at the second wearable device configuration.

In some examples, the control system 106 may be configured to determine, based at least in part on the first heart rate waveforms and the second heart rate waveforms, whether to change a wearable device configuration or maintain a current wearable device configuration. In some such examples, the control system 106 may be configured to make this determination based, at least in part, on a comparison of a first signal-to-noise ratio (SNR) corresponding to the first heart rate waveforms and a second SNR corresponding to the second heart rate waveforms. According to some examples, the control system 106 may be configured to control the user interface system to prompt the user either to change the wearable device configuration or maintain the current wearable device configuration.

According to some examples, the control system 106 may be configured to receive signals from each of a plurality of ultrasonic receiver elements in an array of ultrasonic receiver elements of the ultrasonic receiver system 102. The signals may correspond to the ultrasonic waves generated by the target object responsive to the light from the light source system 104. In some examples, the control system 106 may be configured to apply a receiver-side beamforming process to the ultrasonic receiver signals, to produce a beamformed ultrasonic receiver image. According to some examples, the control system 106 may be configured to detect a blood vessel within the target object based, at least in part, on the beamformed ultrasonic receiver image. In some such examples, the control system 106 may be configured to estimate one or more blood vessel features based, at least in part, on the beamformed ultrasonic receiver image. In some examples, the control system 106 may be configured to estimate one or more cardiac features based, at least in part, on one or more arterial signals, on the blood vessel features. According to some examples, the cardiac features may be, or may include, blood pressure.

In this example, the apparatus 100 has an interface system 108 that includes a user interface system. According to some examples, the user interface system may include a microphone system, a loudspeaker system, a haptic feedback system, a voice command system, one or more displays, or combinations thereof. In some examples, the user interface system may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 108 may include a wireless interface system. In some implementations, the interface system 108 may include one or more network interfaces, one or more interfaces between the control system 106 and a memory system and/or one or more interfaces between the control system 106 and one or more external device interfaces (e.g., ports or applications processors), or combinations thereof.

In some examples, the interface system 108 may include, a force sensor system a pressure sensor system, or both. The force sensor system and/or pressure sensor system (if present) may be, or may include, a piezo-resistive sensor, a capacitive sensor, a thin film sensor (for example, a polymer-based thin film sensor), another type of suitable force sensor, or combinations thereof. If the force sensor system and/or pressure sensor system includes a piezo-resistive sensor, the piezo-resistive sensor may include silicon, metal, polysilicon, glass, or combinations thereof. An ultrasonic fingerprint sensor and a force sensor system and/or pressure sensor system may, in some implementations, be mechanically coupled. In some such examples, the force sensor system and/or pressure sensor system may be integrated into circuitry of the ultrasonic fingerprint sensor. In some examples, the interface system 108 may include an optical sensor system, one or more cameras, or a combination thereof.

According to some examples, the apparatus 100 may include a noise reduction system 110. For example, the noise reduction system 110 may include one or more mirrors that are configured to reflect light from the light source system 104 away from the ultrasonic receiver system 102. In some implementations, the noise reduction system 110 may include one or more sound-absorbing layers, acoustic isolation material, light-absorbing material, light-reflecting material, or combinations thereof. In some examples, the noise reduction system 110 may include acoustic isolation material, which may reside between the light source system 104 and at least a portion of the ultrasonic receiver system 102, on at least a portion of the ultrasonic receiver system 102, or combinations thereof. In some examples, the noise reduction system 110 may include one or more electromagnetically shielded transmission wires. In some such examples, the one or more electromagnetically shielded transmission wires may be configured to reduce electromagnetic interference from circuitry of the light source system 104, receiver system circuitry, or combinations thereof, that is received by the ultrasonic receiver system 102. In some examples, the one or more electromagnetically shielded transmission wires, sound-absorbing layers, acoustic isolation material, light-absorbing material, light-reflecting material, or combinations thereof may be components of the ultrasonic receiver system 102, the light source system 104, or both. Despite the fact that the ultrasonic receiver system 102, the light source system 104 and the noise reduction system 110 are shown in FIG. 1 as being separate elements, such components may nonetheless be regarded as elements of the noise reduction system 110.

The apparatus 100 may be used in a variety of different contexts, many examples of which are disclosed herein. For example, in some implementations a mobile device may include the apparatus 100. In some such examples, the mobile device may be a smart phone. In some implementations, a wearable device may include the apparatus 100, or the apparatus 100 may be a wearable device. The wearable device may, for example, be a bracelet, an armband, a wristband, a watch, a ring, a headband or a patch. Accordingly, in some examples the apparatus 100 may be configured to be worn by, or attached to, a person.

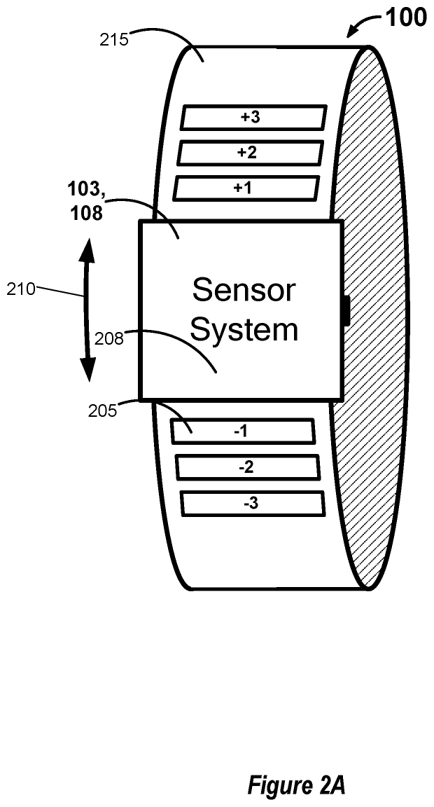

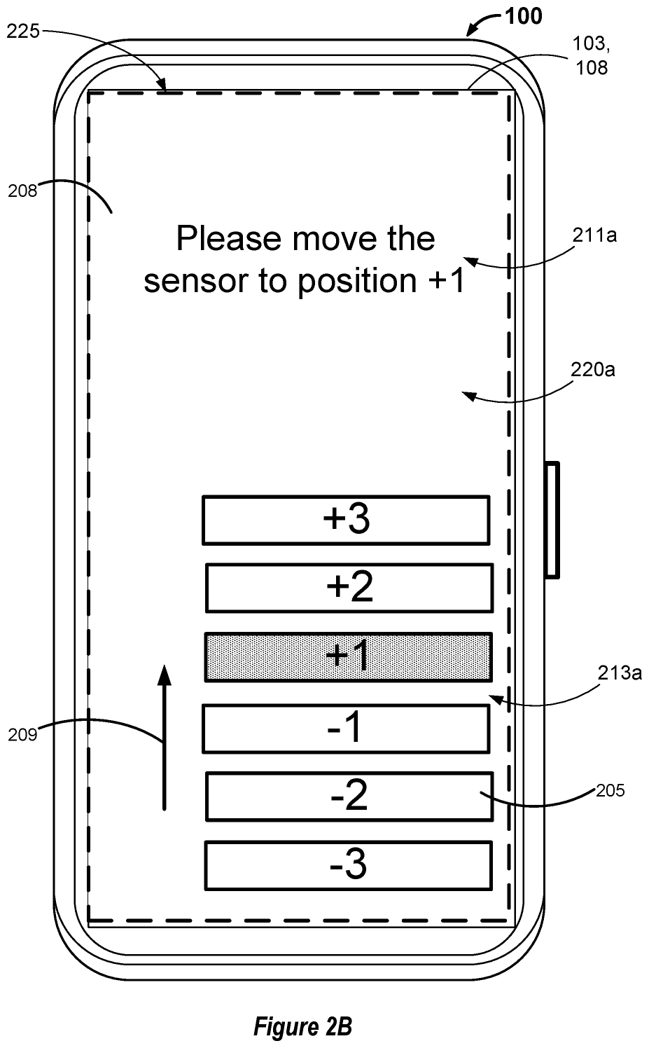

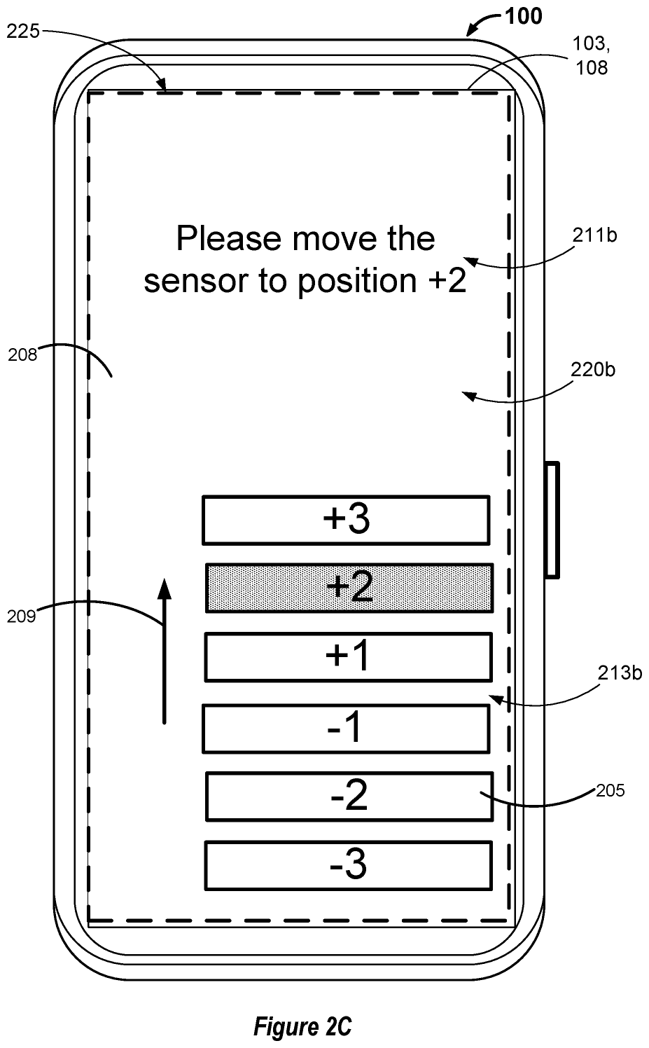

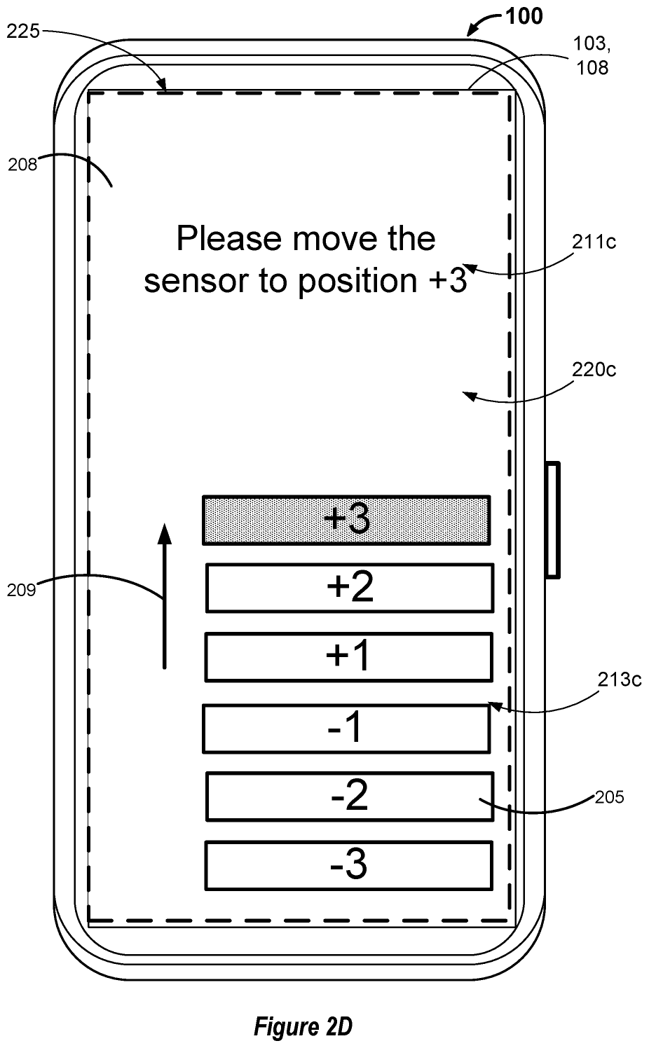

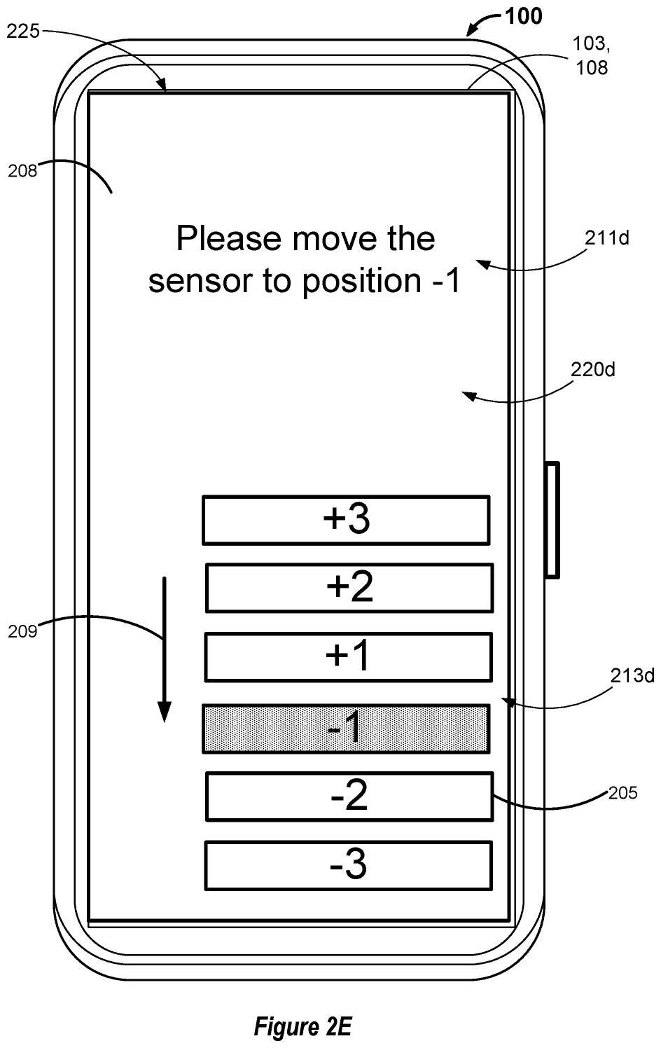

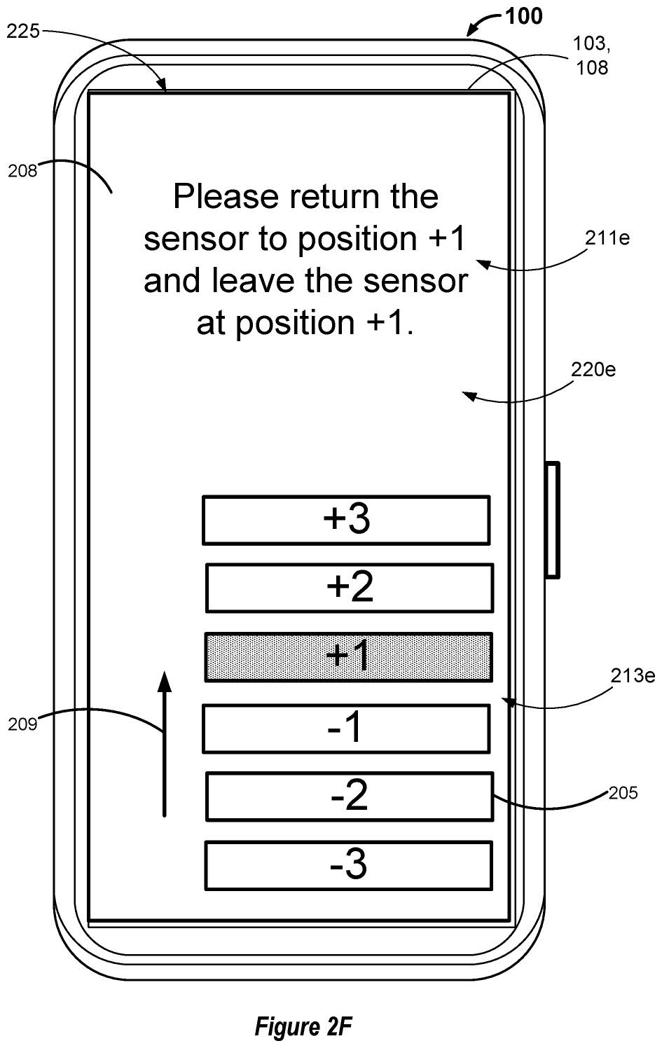

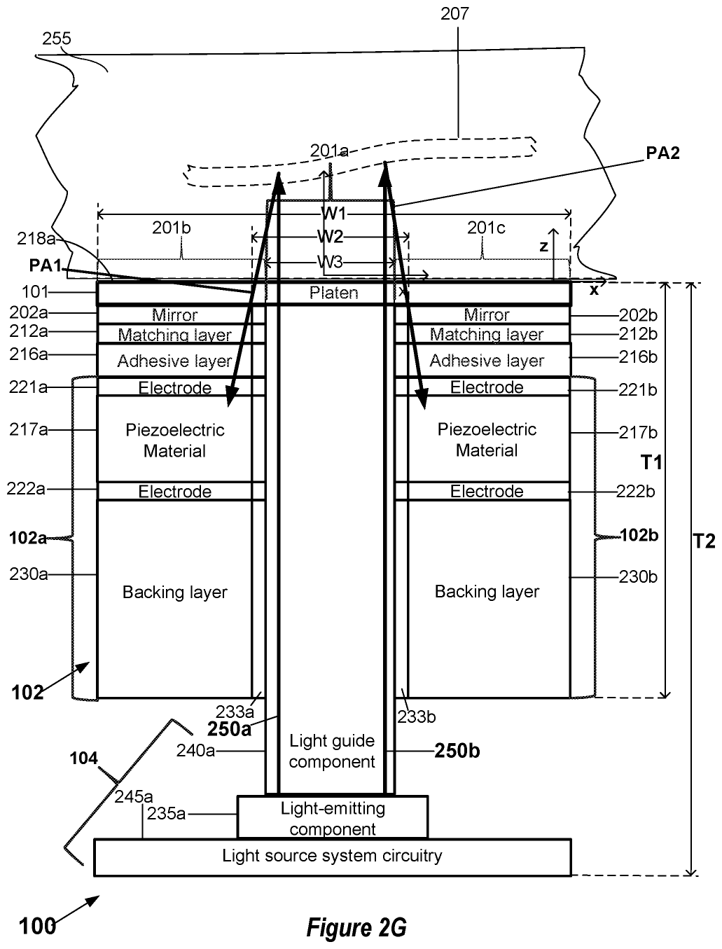

FIG. 2A shows example components of an apparatus according to some disclosed implementations. As with other figures provided herein, the numbers, types and arrangements of elements shown in FIG. 2A are merely presented by way of example. In this example, the apparatus 100 is an instance of the apparatus 100 shown in FIG. 1. According to this example, the apparatus 100 includes a sensor system 103, a control system 106 (not shown in FIG. 2A) and an interface system 108 that includes a user interface system. In this example, the apparatus 100 is a wearable device that includes a strap 215 for securing the apparatus 100 to a person's arm, wrist, ankle, etc. According to some examples, the apparatus 100 may be a smart watch, such as a smart watch that is configured for wireless communication with a cellular telephone.

In this example, the apparatus 100 is shown at a particular wearable device configuration that corresponds with a position of the sensor system 103. According to this example, a user of the apparatus 100 may position the sensor system 103 in any one of seven wearable device configurations: one wearable device configuration—which may be regarded as a “zero position”—is shown in FIG. 2A and the other six wearable device configurations are indicated by the position indicators 205, which show positions −3, −2, −1, +1, +2 and +3. In other implementations of the apparatus 100, the sensor system 103 may be positioned in more or fewer wearable device configurations, such as 2 wearable device configurations, 3 wearable device configurations, 4 wearable device configurations, 5 wearable device configurations, 6 wearable device configurations, 8 wearable device configurations, 9 wearable device configurations, 10 wearable device configurations, etc.

In some alternative examples, only a portion of the sensor system 103 may be positioned in multiple wearable device configurations. In some such examples, the sensor system 103 may include a transmitter portion—such as a light source system, an ultrasonic transmitter system, etc.—that can be positioned in various positions relative to a receiver portion, or vice versa. In some PAPG-enabled examples, the light source system 104 of FIG. 1 may be positioned in various positions relative to the ultrasonic receiver system 102. Each position corresponds to a wearable device configuration.

In some examples, the control system 106 may be configured to obtain, via the sensor system 103, heart rate waveforms at each of a plurality of wearable device configurations. In the example shown in FIG. 2A, the control system 106 is configured to obtain, via the sensor system 103, heart rate waveforms at each of the seven possible wearable device configurations. According to some examples, the control system 106 may be configured to obtain heart rate waveforms at each of a plurality of wearable device configurations and then select a wearable device configuration according to the heart rate waveforms. For example, the control system 106 may be configured to determine a SNR corresponding to each of the heart rate waveforms and to select the wearable device configuration according to the SNRs. In some such examples, the control system 106 may be configured to select the wearable device configuration corresponding to the highest SNR.

In other examples, the control system 106 may be configured to obtain signals from the sensor system 103 at each of a plurality of wearable device configurations corresponding to one or more other targets of interest and to select a wearable device configuration according to the signals corresponding to the one or more other targets of interest. The one or more other targets of interest may, for example, include one or more blood vessel walls, such as one or more arterial walls. For example, the control system 106 may be configured to determine a SNR corresponding to each of the signals corresponding to the one or more other targets of interest and to select the wearable device configuration according to the SNRs. In some such examples, the control system 106 may be configured to select the wearable device configuration corresponding to the highest SNR.

According to this example, the control system 106 is configured to control a user interface of the interface system 108 to provide user prompts. In some examples, the prompts may be, or may include, visual prompts made via one or more displays of the interface system 108, such as the display 208. Alternatively, or additionally, the prompts may be, or may include, audio prompts made via one or more loudspeakers of the interface system 108. Some such user prompts may be, or may include, user prompts either to change the current wearable device configuration or maintain the current wearable device configuration. In some examples, the user prompts may be prompts to change the current wearable device configuration to another wearable device configuration, to allow the control system 106 to obtain signals from the sensor system 103 at the wearable device configuration.

FIGS. 2B, 2C, 2D, 2E and 2F show examples of graphical user interfaces (GUIs) that may be presented via a display system of an apparatus according to some disclosed implementations. As with other figures provided herein, the numbers, types and arrangements of elements shown in FIGS. 2B-2F are merely presented by way of example. In these examples, the GUIs are being presented on a display 208 of an apparatus 100, which is an instance of the apparatus 100 that is shown in FIGS. 1 and 2A. In these examples, the display 208 is a component of the interface system 108 that is shown in FIGS. 1 and 2A. According to these examples, the apparatus 100 also includes a touch sensor system 225, which is also a component of the interface system 108 that is shown in FIGS. 1 and 2A.

FIGS. 2B-2F show examples of user prompts that the control system 106 (not shown) may cause the display 208 to present. In some examples, the prompts may include audio prompts made via one or more loudspeakers of the interface system 108. In these examples, the user prompts are prompts to change the current wearable device configuration to another wearable device configuration, to allow the control system 106 to obtain signals from the sensor system 103 at the next wearable device configuration.

FIG. 2B shows an example of a user prompt that the control system 106 may cause to be presented on the display 208 when the apparatus 100 is in the wearable device configuration shown in FIG. 2A, or when the apparatus 100 is in the wearable device configuration corresponding with the position −1, −2 or −3 shown in FIG. 2A. According to this example, the GUI 220a includes a textual prompt 211a and position change information 213a. In this example, the textual prompt 211a instructs the user to move the sensor 103 to the position +1. The position change information 213a includes the arrow 209, which indicates the direction in which the sensor system 103 should be moved, as well as representations of the positions 205 that are provided on the apparatus 100 that is shown in FIG. 2A. In this example, the position change information 213a displays the position +1 with a patterned background in order to indicate that position +1 is the desired position.

In some examples, before causing the display 208 to present the GUI 220a, the control system 106 may already have obtained, via the sensor system 103, heart rate waveforms—or signals corresponding to the one or more other targets of interest—at one or more other wearable device configurations, such as the wearable device configuration shown in FIG. 2A. According to this example, after causing the display 208 to present the GUI 220a, and after the apparatus is in the wearable device configuration corresponding to position +1, the control system 106 is configured to obtain, via the sensor system 103, additional heart rate waveforms—or additional signals corresponding to the one or more other targets of interest—at the wearable device configuration corresponding to position +1. In some examples, after obtaining data at the wearable device configuration corresponding position +1, the control system 106 may determine that enough information has been required to select a wearable device configuration. For example, the control system may determine that a peak SNR was obtained at a previous wearable device configuration. The peak SNR may, in some examples, be the peak SNR of individual photoacoustic signals from the sensor system 103. In some such examples, the photoacoustic signals may be “raw” signals, on which little or no pre-processing has been perform prior to the SNR evaluation. Alternatively, or additionally, the peak SNR of processed signals, such as filtered signals, beamformed signals, heart rate waveforms constructed from received signals, etc., may be evaluated by the control system 106.

However, in these examples, after causing the display 208 to present the GUI 220a and after obtaining data from the sensor system 103 at the wearable device configuration corresponding position +1, the control system 106 causes the display 208 to present the GUI 220b that is shown in FIG. 2C. According to this example, the GUI 220b includes a textual prompt 211b and position change information 213b. In this example, the textual prompt 211b instructs the user to move the sensor 103 to the position +2. According to this example, the position change information 213b displays the position +2 with a patterned background in order to indicate that position +2 is the desired position.

According to this example, after causing the display 208 to present the GUI 220b, and after the apparatus is in the wearable device configuration corresponding to position +2, the control system 106 is configured to obtain, via the sensor system 103, additional heart rate waveforms—or signals corresponding to one or more other targets of interest—at the wearable device configuration corresponding to position +2. In some examples, after obtaining data at the wearable device configuration corresponding position +2, the control system 106 may determine that enough information has been required to select a wearable device configuration.

However, in these examples, after causing the display 208 to present the GUI 220b and after obtaining data at the wearable device configuration corresponding position +2, the control system 106 causes the display 208 to present the GUI 220c that is shown in FIG. 2D. According to this example, the textual prompt 211c instructs the user to move the sensor 103 to the position +3 and the position change information 213c displays the position +3 with a patterned background in order to indicate that position +3 is the desired position.

According to this example, after causing the display 208 to present the GUI 220c, and after the apparatus is in the wearable device configuration corresponding to position +3, the control system 106 is configured to obtain, via the sensor system 103, additional heart rate waveforms—or signals corresponding to the one or more other targets of interest—at the wearable device configuration corresponding to position +3. In some examples, after obtaining data at the wearable device configuration corresponding position +3, the control system 106 may determine that enough information has been required to select a wearable device configuration.

However, in these examples, after causing the display 208 to present the GUI 220c and after obtaining data at the wearable device configuration corresponding position +3, the control system 106 causes the display 208 to present the GUI 220d that is shown in FIG. 2E. According to this example, the textual prompt 211d instructs the user to move the sensor 103 to the position −1 and the position change information 213d displays the position −1 with a patterned background in order to indicate that position −1 is the desired position.

According to this example, after causing the display 208 to present the GUI 220d, and after the apparatus is in the wearable device configuration corresponding to position −1, the control system 106 is configured to obtain, via the sensor system 103, additional heart rate waveforms—or signals corresponding to the one or more other targets of interest—at the wearable device configuration corresponding to position −1. In this example, after obtaining data at the wearable device configuration corresponding position −1, the control system 106 determines that enough information has been required to select a wearable device configuration.

In this example, the control system selects the wearable device configuration corresponding to position +1. For example, the control system may determine that a peak SNR was obtained at the wearable device configuration corresponding to position +1. Accordingly, the control system 106 causes the display 208 to present the GUI 220c that is shown in FIG. 2F. According to this example, the textual prompt 211e instructs the user to return the sensor 103 to the position +1 and to leave the sensor 103 at the position +1. The position change information 213e displays the position +1 with a patterned background in order to indicate that position +1 is the desired position.

FIG. 2G shows example components of an apparatus according to some disclosed implementations. As with other figures provided herein, the numbers, types and arrangements of elements shown in FIG. 2G are merely presented by way of example. In this example, the apparatus 100 is an instance of the apparatus 100 shown in FIG. 1. According to this example, the apparatus 100 includes a platen 101, a receiver system 102 and a light source system 104. In this example, an outer surface 218a of the platen 101 is configured to receive a target object, such as the finger 255, a wrist, etc.

According to this example, the receiver system 102, is, or includes, an ultrasonic receiver system. In this example, the receiver system 102 includes the receiver stack portion 102a and the receiver stack portion 102b. In this example, the receiver stack portion 102a includes piezoelectric material 217a, an electrode layer 220a on a first side of the piezoelectric material 217a and an electrode layer 222a on a second side of the piezoelectric material 217a. According to some examples, a layer of anisotropic conductive film (ACF) may reside between each of the electrode layers 220a and 220b and the piezoelectric material 217a. In this example, the electrode layer 222a resides between the piezoelectric material 217a and a backing layer 230a. The electrode layers 220a and 220b include conductive material, which may be, or may include, a conductive metal such as copper in some instances. The electrode layers 220a and 220b may be electrically connected to receiver system circuitry, which is not shown in FIG. 2G. The receiver system circuitry may be regarded as a portion of the control system 106 that is described herein with reference to FIG. 1, as a part of the receiver system 102, or both. The piezoelectric material 217a may, for example, include a polyvinylidene difluoride (PVDF) polymer, a polyvinylidene fluoride-trifluoroethylene (PVDF-TrFE) copolymer, aluminum nitride (AlN), lead zirconate titanate (PZT), piezoelectric composite material, such as a 1-3 composite, a 2-2 composite, a 3-3 composite, etc., or combinations thereof. The backing layer 230a may be configured to suppress at least some acoustic artifacts and may provide a relatively higher signal-to-noise ratio (SNR) than receiver systems 102 that lack a backing layer. In some examples, the backing layer 230a may include metal, epoxy, or a combination thereof.

In this example, the receiver stack portion 102b includes piezoelectric material 217b, an electrode layer 220b on a first side of the piezoelectric material 217b and an electrode layer 222b on a second side of the piezoelectric material 217b. Here, the electrode layer 222b resides between the piezoelectric material 217b and a backing layer 230b. According to this example, the receiver stack portion 102a resides proximate a first side of the light guide component 240a and the receiver stack portion 102b resides proximate a second side of the light guide component 240a. In this example, the piezoelectric materials 216a and 216b are configured to produce electric signals in response to received acoustic waves, such as the photoacoustic waves PA1 and PA2.

According to this example, the light source system 104 includes at least a first light-emitting component (the light-emitting component 235a in this example), at least a first light guide component (the light guide component 240a in this example) and light source system circuitry 245a. The light-emitting component 235a may, for example, include one or more light-emitting diodes, one or more laser diodes, one or more VCSELs, one or more edge-emitting lasers, one or more neodymium-doped yttrium aluminum garnet (Nd:YAG) lasers, or combinations thereof.

The light guide component 240a may include any suitable material, or combination of materials, for causing at least some of the light emitted by the light-emitting component 235a to propagate within the light guide component 240a, for example due to total internal reflection between one or more core materials and one or more cladding materials of the light guide component 240a. In such examples, the core material(s) will have a higher index of refraction than the cladding material(s). In one specific and non-limiting example, the core material may have an index of refraction of approximately 1.64 and the cladding material may have an index of refraction of approximately 1.3. In some examples, the core material(s) may include glass, silica, quartz, plastic, zirconium fluoride, chalcogenide, or combinations thereof. According to some examples, the cladding material(s) may include polyvinyl chloride (PVC), acrylic, polytetrafluoroethylene (PTFE), silicone or fluorocarbon rubber. The light guide component 240a may, in some examples, include one or more optical fibers. As used herein, the terms “light guide” and “light pipe” may be used synonymously.

In some examples, the width W3 of the light guide component 240a may be in the range of 0.25 mm to 3 mm, for example 0.5 mm, 1.0 mm, 1.5 mm, etc. According to some examples, the width W2 of the space between the receiver stack portion 102a and the receiver stack portion 102b may be in the range of 0.5 mm to 5 mm, for example 1.0 mm, 1.5 mm, 2 mm, 2.5 mm, etc. In some examples, the space 233a between the receiver stack portion 102a and the light guide component 240a and the space 233b between the receiver stack portion 102b and the light guide component 240a, if any—in other words, the space(s) between W2 and W3, if any—may include light-absorbing material. According to some examples, the spaces 233a and 233b, if any, may include air. In some examples, the spaces 233a and 233b, if any, may include sound-absorbing material, preferably sound-absorbing material having a relatively low Grüneisen parameter.

In this example, the light source system 104 is configured to emit light through a first area of the platen towards a target object that is in contact with the first area of the platen 101. According to this example, the light source system 104 is configured to transmit light-represented in FIG. 2G by the light rays 250a and 250b-through the light guide component 240a and the platen area 201a towards the finger 255, which is in contact with the platen area 201a. In this example, an arterial wall of the artery 207 produces the photoacoustic waves PA1 and PA1 responsive to the light rays 250a and 250b, respectively.

The platen 101 may include any suitable material, such as glass, acrylic, polycarbonate, combinations thereof, etc. In some examples, the width W1 of the platen 101 may be in the range of 2 mm to 10 mm, for example 4 mm, 5 mm, 6 mm, etc. According to some examples, the thickness of the platen 101 (in the z direction of the coordinate system shown in FIG. 2G) may be in the range of 50 microns to 500 microns, for example 150 microns, 200 microns, 250 microns, 300 microns, etc.

In this example, the platen 101 includes platen areas 201a, 201b and 201c. In this example, the platen area 201a resides adjacent the light guide component 240a. Accordingly, at least the platen area 201a includes transparent material in this example. According to some examples, the platen 101 may include one or more anti-reflective layers. In some examples, one or more anti-reflective layers may reside on the platen 101, or proximate the platen 101, for example on or proximate the outer surface 218a.

According to this example, the platen area 201b resides proximate the receiver stack portion 102a and the platen area 201c resides proximate the receiver stack portion 102c. In this example, a mirror layer 202a, a matching layer 212a and an adhesive layer 216a reside between the platen area 201b and the receiver stack portion 102a. Similarly, in this example a mirror layer 202b, a matching layer 212b and an adhesive layer 216b reside between the platen area 201c and the receiver stack portion 102b. The matching layers 212a and 212b may have an acoustic impedance that is selected to reduce the reflections of acoustic waves caused by the acoustic impedance contrast between one or more layers of the receiver stack portions 102a and 102b that are adjacent to, or proximate, the matching layers 212a and 212b. According to some examples, the matching layers 212a and 212b may include polyethylene terephthalate (PET). In some examples, the adhesive layers 216a and 216b may include pressure-sensitive adhesive (PSA) material.

In the example shown in FIG. 2G, the apparatus has a thickness (along the z axis) of T1 from the top of the platen to the base of the backing layers 230a and 230b, and has a thickness of T2 from the top of the platen to the base of the light source system circuitry. In some examples, T2 may be in the range of 2 mm to 10 mm. According to some examples, T1 may be in the range of 1 mm to 8 mm. The backing layers 230a and 230b may be in the range of 3 mm to 7 mm in thickness, such as 4.5 mm, 5.0 mm, 5.5, mm, etc. Accordingly, implementations that lack a backing layer, or backing layers, may be substantially thinner than implementations that include a backing layer, or backing layers.

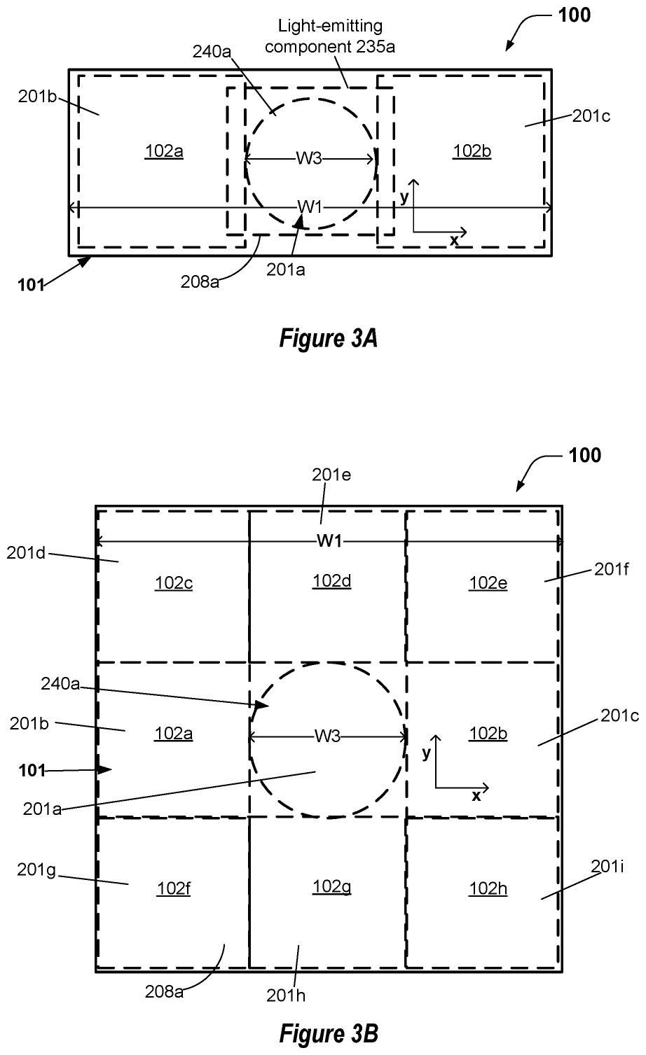

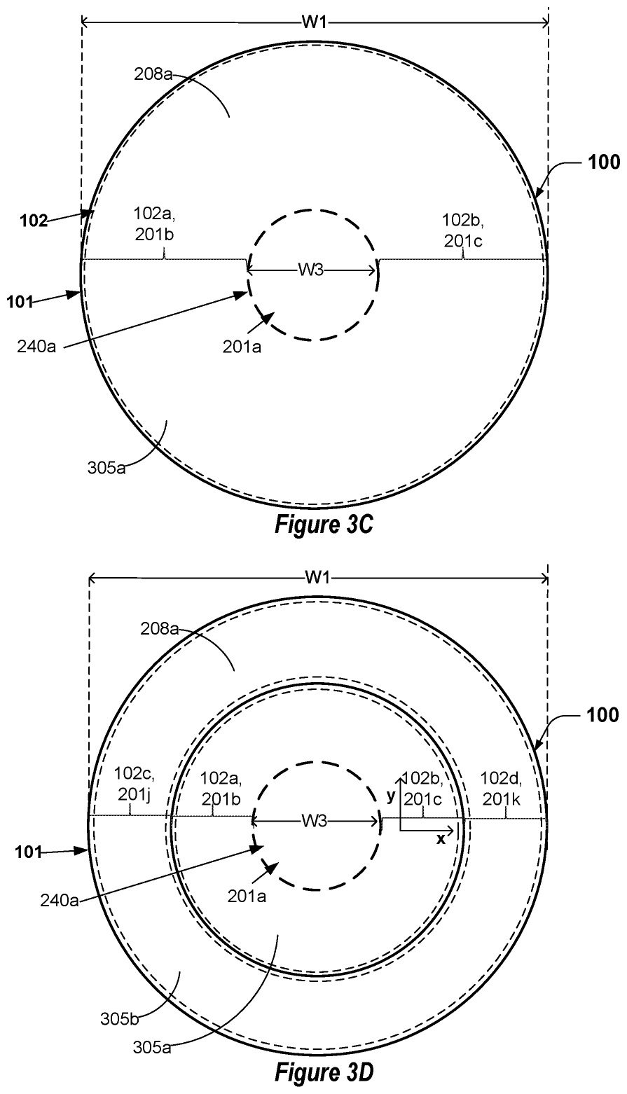

FIGS. 3A, 3B and 3C show different examples of how some components of the apparatus shown in FIG. 2G may be arranged. As with other figures provided herein, the numbers, types and arrangements of elements shown in FIGS. 3A-3C are merely presented by way of example. In these examples, the apparatus 100 is an instance of the apparatus 100 shown in FIGS. 1 and 2G. In each of these examples, a top view of the apparatus 100 is shown, with the view being along the z axis of the coordinate system shown in FIG. 2G. In these examples, the light guide component 240a is shown to have a circular cross-section. However, in alternative examples the light guide component 240a may have a different cross-sectional shape, such as a square cross-sectional shape, a rectangular cross-sectional shape, a hexagonal cross-sectional shape, etc.

In these examples, the outlines of the receiver stack portion 102a and the receiver stack portion 102b (and, in FIG. 3B, the outlines of the receiver stack portions 102c-102h) are shown in dashes, indicating that these elements are below the outer surface 218a of the platen 101. According to these examples, the receiver stack portion 102a resides proximate a first side of the light guide component 240a and the receiver stack portion 102b resides proximate a second side of the light guide component 240a. In these examples, the receiver stack portion 102a resides proximate (in this example, below, further away from the viewer along the z axis) platen area 102b on a first side of the platen area 102a and the receiver stack portion 102b resides proximate platen area 102c, which is on a second and opposite side of the platen area 102a.

According to the example shown in FIG. 3A, the receiver stack portion 102a and the receiver stack portion 102b are discrete elements of a linear array of receiver stack portions having N receiver elements, with N being 2 in this instance. In alternative examples, N may be greater than 2.

In the example shown in FIG. 3B, the receiver stack portion 102a and the receiver stack portion 102b are discrete elements of a two-dimensional receiver array of receiver stack portions having M receiver elements, with M being 9 in this instance. In alternative examples, M may be greater than or less than 9.

According to the example shown in FIG. 3C, the receiver stack portion 102a and the receiver stack portion 102b are portions of a receiver stack ring 305a. In this example, the receiver stack ring 305a is configured to surround the light guide component 240a. According to this example, an annular area of the platen 301 proximate (in this example, above, closer to the viewer along the z axis) the receiver stack ring 305a, which includes the platen areas 201b and 201c, is configured to surround the platen area 201a.

FIG. 3D shows examples of the components of the apparatus shown in FIG. 2G arranged with additional components. As with other figures provided herein, the numbers, types and arrangements of elements shown in FIG. 3D are merely presented by way of example. In these examples, the apparatus 100 is an instance of the apparatus 100 shown in FIG. 1. In this example, a top view of the apparatus 100 is shown, with the view being along the z axis of the coordinate system shown in FIG. 2G. In this example, the light guide component 240a is shown to have a circular cross-section. However, in alternative examples the light guide component 240a may have a different cross-sectional shape.

In this example, the receiver stack portion 102a and the receiver stack portion 102b are portions of a receiver stack ring 305a. According to this example, the receiver stack ring 305a is configured to surround the light guide component 240a. In this example, the receiver stack ring 305a includes the receiver stack portions 102a and 102b, as well as the platen areas 201b and 201c. According to this example, the receiver stack ring 305b is configured to surround the receiver stack ring 305a. In this example, the receiver stack ring 305b includes the receiver stack portions 102c and 102d, as well as the platen areas 201j and 201k.

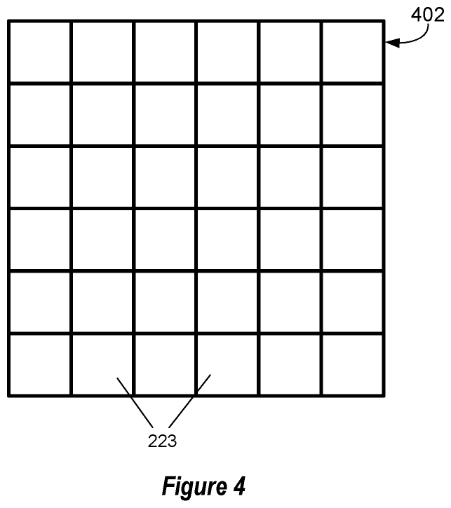

FIG. 4 shows another example of an ultrasonic receiver element array. In this example, the array of ultrasonic receiver elements 402 is a two-dimensional array of ultrasonic receiver elements. According to this example, the array of ultrasonic receiver elements 402 is arranged in a square having 6 active ultrasonic receiver elements 223 on each side and a total of 36 active ultrasonic receiver elements 223. Each row and column of the array of ultrasonic receiver elements 402 may be considered a linear array having 6 active ultrasonic receiver elements 223. As with other disclosed examples, the type, number, size and arrangement of elements shown in FIG. 4 and described herein are merely examples. For example, alternative examples of two-dimensional arrays of ultrasonic receiver elements may be arranged in a different shape, such as a non-square rectangular shape, a hexagonal shape, etc. Some alternative examples of two-dimensional arrays of ultrasonic receiver elements may include a different number of active ultrasonic receiver elements 223, such as 16, 20, 25, 30, 32, 36, 40, 48, etc.

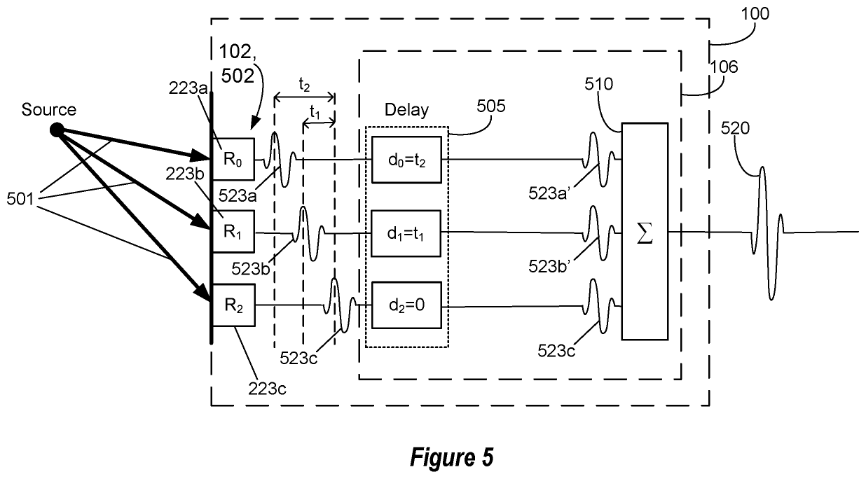

FIG. 5 shows an example of an apparatus that is configured to perform a receiver-side beamforming process. In this example, the receiver-side beamforming process is a delay-and-sum beamforming process. As with other disclosed examples, the types, numbers, sizes and arrangements of elements shown in FIG. 5 and described herein, as well as the associated described methods, are merely examples.

In this example, a source is shown emitting ultrasonic waves 501, which are detected by active ultrasonic receiver elements 223a, 223b and 223c of an array of ultrasonic receiver elements 502. The array of ultrasonic receiver elements 502 is part of an ultrasonic receiver system 102. The ultrasonic waves 501 may, in some examples, correspond to the photoacoustic response of a target object to light emitted by a light source system 104 of the apparatus 100. In this example, the active ultrasonic receiver elements 223a, 223b and 223c provide ultrasonic receiver signals 523a, 523b and 523c, respectively, to the control system 106.

According to this example, the control system 106 includes a delay module 505 and a summation module 510. In this example, the delay module 505 is configured to determine whether a delay should be applied to each of the ultrasonic receiver signals 523a. 523b and 523c, and if so, what delay to apply. According to this example, the delay module 505 determines that a delay d₀ of t₂ should be applied to the ultrasonic receiver signal 523a, that a delay d₁ of t₁ should be applied to the ultrasonic receiver signal 523b and that no delay should be applied to the ultrasonic receiver signal 523c. Accordingly, the delay module 505 applies a delay of t₂ to the ultrasonic receiver signal 523a, producing the ultrasonic receiver signal 523a′, and applies a delay of t₁ to the ultrasonic receiver signal 523b, producing the ultrasonic receiver signal 523b′.

In some examples, the delay module 505 may determine what delay, if any, to apply to an ultrasonic receiver signal by performing a correlation operation on input ultrasonic receiver signals. For example, the delay module 505 may perform a correlation operation on the ultrasonic receiver signals 523a and 523c, and may determine that by applying a time shift of t₂ to the ultrasonic receiver signal 523a, the ultrasonic receiver signal 523a would be strongly correlated with the ultrasonic receiver signal 523c. Similarly, the delay module 505 may perform a correlation operation on the ultrasonic receiver signals 523b and 523c, and may determine that by applying a time shift of t₁ to the ultrasonic receiver signal 523b, the ultrasonic receiver signal 523b would be strongly correlated with the ultrasonic receiver signal 523c.

According to this example, the summation module 510 is configured to sum the ultrasonic receiver signals 523a′, 523b′ and 523c, producing the summed signal 520. One may observe that the amplitude of the summed signal 520 is greater than the amplitude of any one of the ultrasonic receiver signals 523a, 523b or 523c. In some instances, the signal-to-noise ratio (SNR) of the summed signal 520 may be greater than the SNR of any of the ultrasonic receiver signals 523a, 523b or 523c.

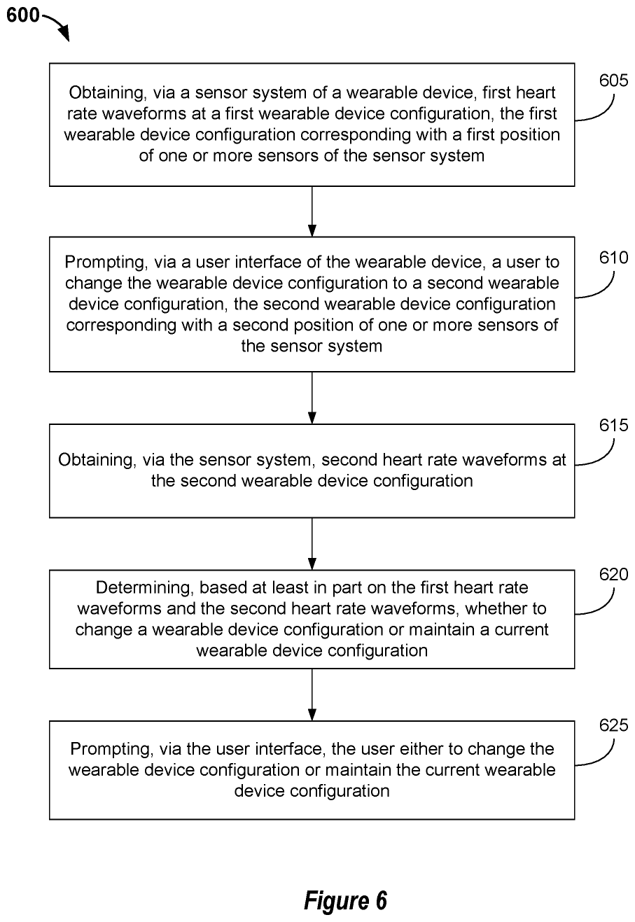

FIG. 6 is a flow diagram that shows examples of some disclosed operations. The blocks of FIG. 6 may, for example, be performed by the apparatus 100 of FIG. 1, by the apparatus 100 of FIG. 2, or by a similar apparatus. As with other methods disclosed herein, the method outlined in FIG. 6 may include more or fewer blocks than indicated. Moreover, the blocks of methods disclosed herein are not necessarily performed in the order indicated. In some instances, one or more of the blocks shown in FIG. 6 may be performed concurrently.

In this example, block 605 involves obtaining, via a sensor system of a wearable device, first heart rate waveforms at a first wearable device configuration. According to this example, the first wearable device configuration corresponds with a first position of one or more sensors of the sensor system. The first heart rate waveforms may, for example, be obtained from a target object such as a finger, a wrist, an ankle, etc., depending on the particular implementation. In some alternative examples, block 605—or another aspect of method 600—may involve obtaining signals corresponding to one or more other targets of interest, such as signals corresponding to at least a portion of a blood vessel, corresponding to blood within a blood vessel, etc.

According to this example, block 610 involves prompting, via a user interface of the wearable device, a user to change the wearable device configuration to a second wearable device configuration. In this example, the second wearable device configuration corresponds with a second position of one or more sensors of the sensor system. Block 610 may, for example, involve presenting one or more visual prompts, such as one of the visual prompts that are illustrated in FIGS. 2B-2E, presenting one or more audio prompts, or combinations thereof.

According to this example, block 615 involves obtaining, via the sensor system, second heart rate waveforms at the second wearable device configuration. In some alternative examples, block 615 may involve obtaining signals corresponding to one or more other targets of interest.

In this example, block 620 involves determining, based at least in part on the first heart rate waveforms and the second heart rate waveforms, whether to change a wearable device configuration or maintain a current wearable device configuration. In some alternative examples, block 620 may involve determining, based at least in part on first and second instances of signals corresponding to one or more other targets of interest, whether to change the wearable device configuration or maintain a current wearable device configuration. In some examples, block 620—or another aspect of method 600—may involve determining a first signal-to-noise ratio (SNR) corresponding to the first heart rate waveforms (or other signals of interest), determining a second SNR corresponding to the second heart rate waveforms (or other signals of interest), and comparing the first SNR to the second SNR.

According to this example, block 625 involves prompting, via the user interface, the user either to change the wearable device configuration or maintain the current wearable device configuration.

In some examples, method 600 may involve obtaining, via the sensor system, first through N^{th} heart rate waveforms (or first through N^{th} instances of other signals of interest) at first through N^{th} wearable device configurations, N being an integer greater than 2. In some such examples, method 600 may involve selecting, based at least in part on the first through N^{th} heart rate waveforms (or first through N^{th} instances of other signals of interest), one of the first through N^{th} wearable device configurations. In some such examples, method 600 may involve prompting, via the user interface, the user to change the wearable device configuration to a selected wearable device configuration.

According to some examples, the heart rate waveforms (or the other signals of interest) may be obtained via a photoacoustic process. In some such examples, the photoacoustic process may involve controlling a light source system of the wearable device to direct light to a target portion of a user's body and obtaining, via a receiver system of the wearable device, acoustic signals corresponding to photoacoustic responses of the target portion of the user's body to the light. FIG. 2G and the corresponding description provide examples.

In some examples, method 600 may involve obtaining, via the sensor system, sensor data at a current pressure being applied to a user's body by the wearable device at a current wearable device configuration. The sensor data may, for example, be obtained by one or more pressure sensors of the sensor system 103. The current pressure may, for example, correspond to a tightness of a strap, a belt, or a similar mechanism for securing the apparatus 100 to a person's arm, wrist, ankle, etc., such as the strap 215 that is shown in FIG. 2A. In some examples, method 600 may involve determining, based at least in part on the sensor data, whether to prompt the user to change the current pressure to another pressure. For example, method 600 may involve determining, based at least in part on the sensor data, whether to prompt the user to tighten or loosen the strap 215. According to some examples, determining whether to prompt the user to change the current pressure to another pressure may involve determining whether the current pressure is within a desired pressure range.

According to some examples, determining whether to prompt the user to change the current pressure to another pressure may involve determining whether blood vessel distension has been reduced at the current pressure. For example, if the user has tightened the strap 215 (in some instances, pursuant to a prompt caused by the control system) and if the control system determines that a sensor data obtained after tightening indicates that blood vessel distension has been reduced at the current pressure, as compared to previously-measured blood vessel distension based on signals obtained at a lower pressure, the control system may cause the interface system to provide a user prompt to loosen the strap 215.

In some examples, a user may use a hand, such as the right hand, to change the configuration of a wearable device that is worn on the left wrist or elsewhere on a user's body. In some such instances, if the user leaves the hand used to change the configuration of the wearable device on or proximate the wearable device while the wearable device is obtaining heart rate waveforms or other signals, the proximity of the hand may cause interference. Therefore, in some examples method 600 may involve prompting, via the user interface, the user to withdraw a hand used to change the wearable device configuration.

In some examples, method 600 may involve estimating one or more blood vessel features based, at least in part, on signals obtained from the sensor system 103. The one or more blood vessel features may, for example, include blood vessel diameter, blood vessel area, blood vessel profile, blood vessel distention, volumetric flow, pulse wave velocity, blood vessel wall thickness, or combinations thereof. The one or more blood vessel features may, in some examples, be arterial features. In some examples, method 600 may involve estimating one or more cardiac features based, at least in part, on the one or more blood vessel features. According to some such examples, method 600 may involve estimating blood pressure based, at least in part, on the one or more blood vessel features.

In some examples, method 600 may involve estimating blood pressure based, at least in part, on heart rate waveforms obtained by the sensor system 103. According to some examples, method 600, may involve extracting and evaluating heart rate waveform features.

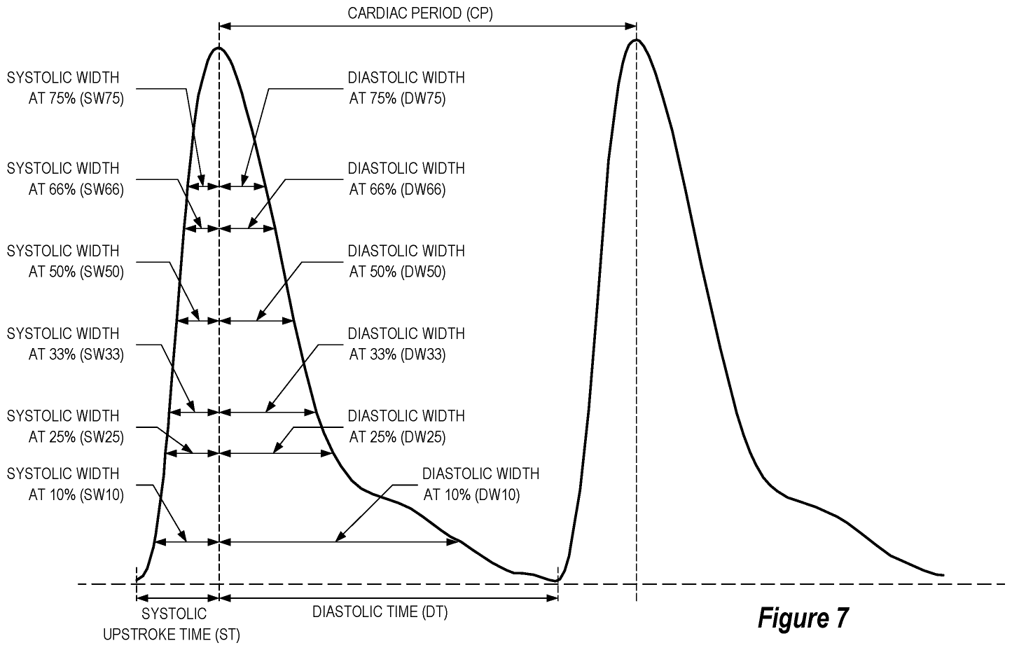

FIG. 7 shows examples of heart rate waveform (HRW) features that may be extracted according to some implementations. The horizontal axis of FIG. 7 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 7 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

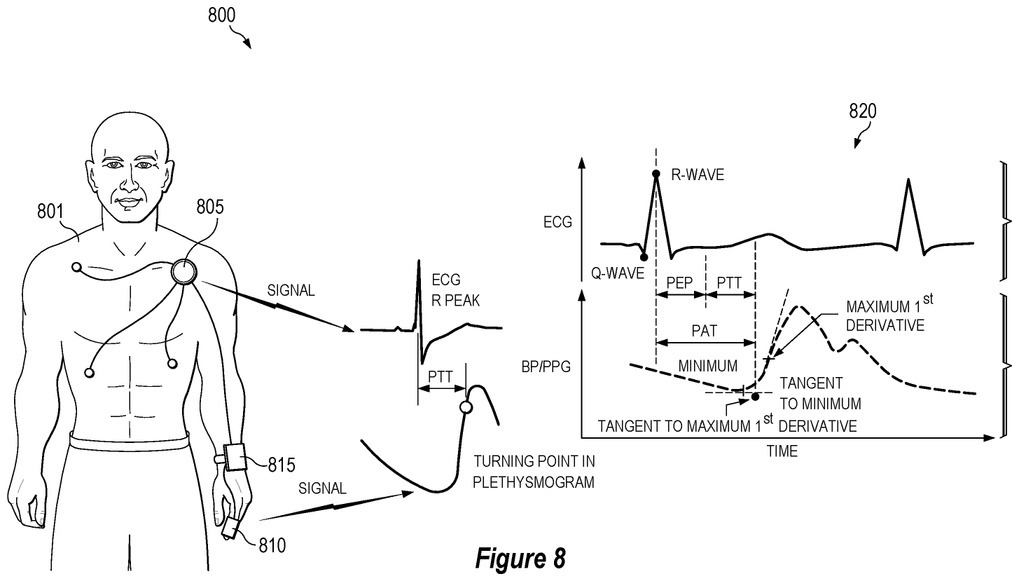

FIG. 8 shows examples of devices that may be used in a system for estimating blood pressure based, at least in part, on pulse transit time (PTT). As with other figures provided herein, the numbers, types and arrangements of elements are merely presented by way of example. According to this example, the system 800 includes at least two sensors. In this example, the system 800 includes at least an electrocardiogram sensor 805 and a device 810 that is configured to be mounted on a finger of the person 801. In this example, the device 810 is, or includes, an apparatus configured to perform at least some PAPG methods disclosed herein. For example, the device 810 may be, or may include, the apparatus 300 of FIG. 3 or a similar apparatus.

As noted in the graph 820, the PAT includes two components, the pre-ejection period (PEP, the time needed to convert the electrical signal into a mechanical pumping force and isovolumetric contraction to open the aortic valves) and the PTT. The starting time for the PAT can be estimated based on the QRS complex—an electrical signal characteristic of the electrical stimulation of the heart ventricles. As shown by the graph 820, in this example the beginning of a pulse arrival time (PAT) may be calculated according to an R-Wave peak measured by the electrocardiogram sensor 805 and the end of the PAT may be detected via analysis of signals provided by the device 810. In this example, the end of the PAT is assumed to correspond with an intersection between a tangent to a local minimum value detected by the device 810 and a tangent to a maximum slope/first derivative of the sensor signals after the time of the minimum value.

There are many known algorithms for blood pressure estimation based on the PTT and/or the PAT, some of which are summarized in Table 1 and described in the corresponding text on pages 5-10 of Sharma, M., et al., Cuff-Less and Continuous Blood Pressure Monitoring: a Methodological Review (“Sharma”), in Multidisciplinary Digital Publishing Institute (MDPI) Technologies 2017, 5, 21, both of which are hereby incorporated by reference.

Some previously-disclosed methods have involved calculating blood pressure according to one or more of the equations shown in Table 1 of Sharma, or other known equations, based on a PTT and/or PAT measured by a sensor system that includes a PPG sensor. As noted above, some disclosed PAPG-based implementations are configured to distinguish artery HRWs from other HRWs. Such implementations may provide more accurate measurements of the PTT and/or PAT, relative to those measured by a PPG sensor. Therefore, disclosed PAPG-based implementations may provide more accurate blood pressure estimations, even when the blood pressure estimations are based on previously-known formulae.

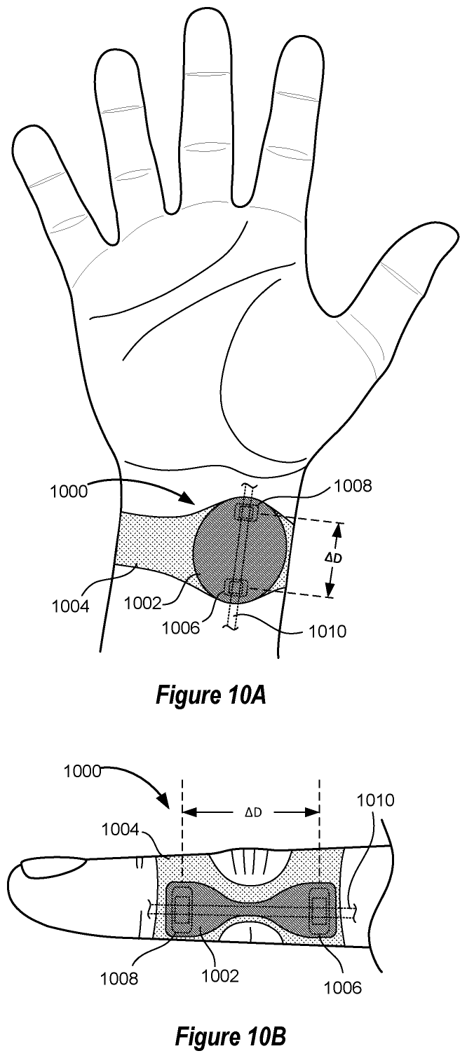

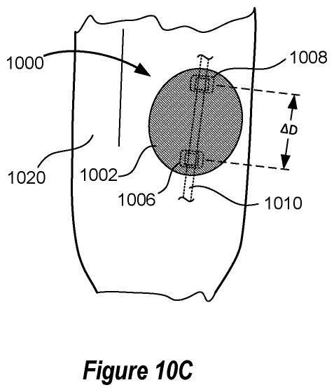

Other implementations of the system 800 may not include the electrocardiogram sensor 805. In some such implementations, the device 815, which is configured to be mounted on a wrist of the person 801, may be, or may include, an apparatus configured to perform at least some PAPG methods disclosed herein. For example, the device 815 may be, or may include, the apparatus 100 of FIG. 2 or a similar apparatus. According to some such examples, the device 815 may include a light source system and two or more ultrasonic receivers. One example is described below with reference to FIG. 10A. In some examples, the device 815 may include an array of ultrasonic receivers.

In some implementations of the system 800 that do not include the electrocardiogram sensor 805, the device 810 may include a light source system and two or more ultrasonic receivers. One example is described below with reference to FIG. 10B.

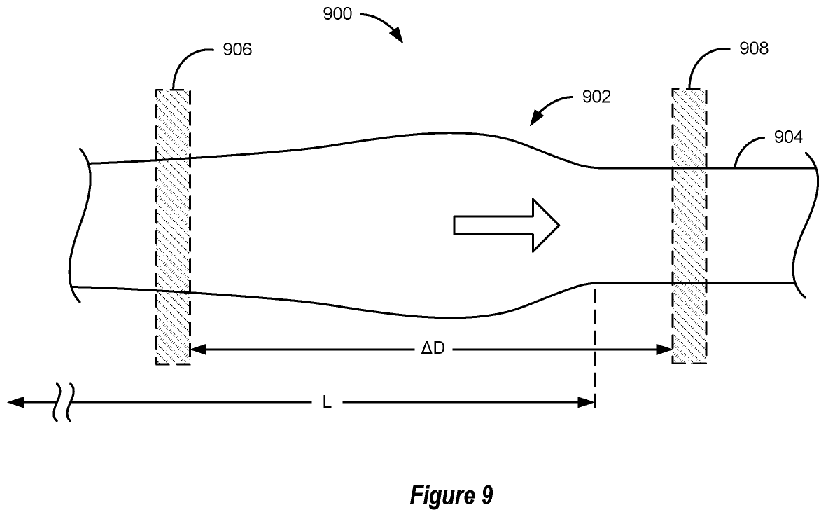

FIG. 9 shows a cross-sectional side view of a diagrammatic representation of a portion of an artery 900 through which a pulse 902 is propagating. The block arrow in FIG. 9 shows the direction of blood flow and pulse propagation. As diagrammatically shown, the propagating pulse 902 causes strain in the arterial walls 904, which is manifested in the form of an enlargement in the diameter (and consequently the cross-sectional area) of the arterial walls—referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length L of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. It will be appreciated that this PTT is different from the PTT that is described above with reference to FIG. 15. However, either version of the PTT may be used for the purpose of blood pressure estimation. Assuming that the physical distance AD between the first and the second physical locations is ascertainable, the PWV can be estimated as the quotient of the physical spatial distance AD traveled by the pulse divided by the time (PTT) the pulse takes in traversing the physical spatial distance AD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance ΔD through which the pulse propagates between the first physical location and the second physical location. More specifically, the PWV generally depends on a number of factors including the density of the blood p, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length ΔD between the two locations where the measurements are performed).

In traditional methods for obtaining PWV, the starting time of the pulse has been obtained at the heart using an electrocardiogram (ECG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 906 and a second arterial distension sensor 908 proximate first and second physical locations, respectively, along an artery of interest. In some particular implementations, the first arterial distension sensor 906 and the second arterial distension sensor 908 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance ΔD of separation between the first arterial distension sensor 906 and the second arterial distension sensor 908 (and consequently the distance between the first and the second locations along the artery) can be in the range of about 1 centimeter (cm) to tens of centimeters—long enough to distinguish the arrival of the pulse at the first physical location from the arrival of the pulse at the second physical location, but close enough to provide sufficient assurance of arterial consistency. In some specific implementations, the distance ΔD between the first and the second arterial distension sensors 906 and 908 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance ΔD between the first and the second arterial distension sensors 906 and 908 can be less than or equal to 1 cm, for example, about 0.1 cm, about 0.25 cm, about 0.5 cm or about 0.75 cm. By way of reference, a typical PWV can be about 15 meters per second (m/s). Using an ambulatory monitoring device in which the first and the second arterial distension sensors 906 and 908 are separated by a distance of about 5 cm, and assuming a PWV of about 15 m/s implies a PTT of approximately 3.3 milliseconds (ms).

The value of the magnitude of the distance ΔD between the first and the second arterial distension sensors 906 and 908, respectively, can be preprogrammed into a memory within a monitoring device that incorporates the sensors (for example, such as a memory of, or a memory configured for communication with, the control system 306 that is described above with reference to FIG. 3). As will be appreciated by a person of ordinary skill in the art, the spatial length L of a pulse can be greater than the distance ΔD from the first arterial distension sensor 906 to the second arterial distension sensor 908 in such implementations. As such, although the diagrammatic pulse 902 shown in FIG. 9 is shown as having a spatial length L comparable to the distance between the first arterial distension sensor 906 and the second arterial distension sensor 908, in actuality each pulse can typically have a spatial length L that is greater and even much greater than (for example, about an order of magnitude or more than) the distance ΔD between the first and the second arterial distension sensors 906 and 908.

### Sensing Architecture and Topology

In some implementations of the ambulatory monitoring devices disclosed herein, both the first arterial distension sensor 906 and the second arterial distension sensor 908 are sensors of the same sensor type. In some such implementations, the first arterial distension sensor 906 and the second arterial distension sensor 908 are identical sensors. In such implementations, each of the first arterial distension sensor 906 and the second arterial distension sensor 908 utilizes the same sensor technology with the same sensitivity to the arterial distension signal caused by the propagating pulses, and has the same time delays and sampling characteristics. In some implementations, each of the first arterial distension sensor 906 and the second arterial distension sensor 908 is configured for photoacoustic plethysmography (PAPG) sensing, e.g., as disclosed elsewhere herein. Some such implementations include a light source system and two or more ultrasonic receivers, which may be instances of the light source system 304 and the receiver system 302 of FIG. 3. In some implementations, each of the first arterial distension sensor 906 and the second arterial distension sensor 908 is configured for ultrasound sensing via the transmission of ultrasonic signals and the receipt of corresponding reflections. In some alternative implementations, each of the first arterial distension sensor 906 and the second arterial distension sensor 908 may be configured for impedance plethysmography (IPG) sensing, also referred to in biomedical contexts as bioimpedance sensing. In various implementations, whatever types of sensors are utilized, each of the first and the second arterial distension sensors 906 and 908 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude- and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension.

In some implementations, regardless of the type of sensors utilized for the first arterial distension sensor 906 and the second arterial distension sensor 908, both the first arterial distension sensor 906 and the second arterial distension sensor 908 can be arranged, assembled or otherwise included within a single housing of a single ambulatory monitoring device. As described above, the housing and other components of the monitoring device can be configured such that when the monitoring device is affixed or otherwise physically coupled to a subject, both the first arterial distension sensor 906 and the second arterial distension sensor 908 are in contact with or in close proximity to the skin of the user at first and second locations, respectively, separated by a distance ΔD, and in some implementations, along a stretch of the artery between which various arterial properties can be assumed to be relatively constant. In various implementations, the housing of the ambulatory monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the ambulatory monitoring device facilitates and enables long-term wearing and monitoring (for example, over days, weeks or a month or more without interruption) of one or more biological characteristics of interest to obtain a better picture of such characteristics over extended durations of time, and generally, a better picture of the user's health.

In some implementations, the ambulatory monitoring device can be positioned around a wrist of a user with a strap or band, similar to a watch or fitness/activity tracker. FIG. 10A shows an example ambulatory monitoring device 1000 designed to be worn around a wrist according to some implementations. In the illustrated example, the monitoring device 1000 includes a housing 1002 integrally formed with, coupled with or otherwise integrated with a wristband 1004. The first and the second arterial distension sensors 1006 and 1008 may, in some instances, each include an instance of the ultrasonic receiver system 102 and a portion of the light source system 104 that are described above with reference to FIG. 1. In this example, the ambulatory monitoring device 1000 is coupled around the wrist such that the first and the second arterial distension sensors 1006 and 1008 within the housing 1002 are each positioned along a segment of the radial artery 1010 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 1006 and 1008 are separated by a fixed distance ΔD. In some other implementations, the ambulatory monitoring device 1000 can similarly be designed or adapted for positioning around a forearm, an upper arm, an ankle, a lower leg, an upper leg, or a finger (all of which are hereinafter referred to as “limbs”) using a strap or band.

FIG. 10B shows an example ambulatory monitoring device 1000 designed to be worn on a finger according to some implementations. The first and the second arterial distension sensors 1006 and 1008 may, in some instances, each include an instance of the ultrasonic receiver 102 and a portion of the light source system 104 that are described above with reference to FIG. 1.

In some other implementations, the ambulatory monitoring devices disclosed herein can be positioned on a region of interest of the user without the use of a strap or band. For example, the first and the second arterial distension sensors 1006 and 1008 and other components of the monitoring device can be enclosed in a housing that is secured to the skin of a region of interest of the user using an adhesive or other suitable attachment mechanism (an example of a “patch” monitoring device).

FIG. 10C shows an example ambulatory monitoring device 1000 designed to reside on an earbud according to some implementations. According to this example, the ambulatory monitoring device 1000 is coupled to the housing of an earbud 1020. The first and second arterial distension sensors 1006 and 1008 may, in some instances, each include an instance of the ultrasonic receiver 102 and a portion of the light source system 104 that are described above with reference to FIG. 1.

Implementation examples are described in the following numbered clauses:
1. A method, including: obtaining, via a sensor system of a wearable device, first heart rate waveforms at a first wearable device configuration, the first wearable device configuration corresponding with a first position of one or more sensors of the sensor system; prompting, via a user interface of the wearable device, a user to change the wearable device configuration to a second wearable device configuration, the second wearable device configuration corresponding with a second position of one or more sensors of the sensor system; obtaining, via the sensor system, second heart rate waveforms at the second wearable device configuration; determining, based at least in part on the first heart rate waveforms and the second heart rate waveforms, whether to change a wearable device configuration or maintain a current wearable device configuration; and prompting, via the user interface, the user either to change the wearable device configuration or maintain the current wearable device configuration.
2. The method of clause 1, where the determining further includes: determining a first signal-to-noise ratio (SNR) corresponding to the first heart rate waveforms; determining a second SNR corresponding to the second heart rate waveforms; and comparing the first SNR to the second SNR.
3. The method of clause 1 or clause 2, where the method further includes: obtaining, via the sensor system, first through Nth heart rate waveforms at first through Nth wearable device configurations, N being an integer greater than 2; selecting, based at least in part on the first through Nth heart rate waveforms, one of the first through Nth wearable device configurations; and prompting, via the user interface, the user to change the wearable device configuration to a selected wearable device configuration.
4. The method of any one of clauses 1-3, where the first heart rate waveforms and the second heart rate waveforms are obtained via a photoacoustic process.
5. The method of clause 4, where the photoacoustic process involves: controlling a light source system of the wearable device to direct light to a target portion of a user's body; and obtaining, via a receiver system of the wearable device, acoustic signals corresponding to photoacoustic responses of the target portion of the user's body to the light.
6. The method of any one of clauses 1-5, further including: obtaining sensor data at a current pressure being applied to a user's body by the wearable device at the current wearable device configuration; and determining, based at least in part on the sensor data, whether to prompt the user to change the current pressure to another pressure.
7. The method of clause 6, where determining whether to prompt the user to change the current pressure to another pressure involves determining whether the current pressure is within a desired pressure range.
8. The method of clause 6 or clause 7, where determining whether to prompt the user to change the current pressure to another pressure involves determining whether a blood vessel distension has been reduced at the current pressure.
9. The method of any one of clauses 1-8, further including prompting, via the user interface, the user to withdraw a hand used to change the wearable device configuration.
10. The method of any one of clauses 1-9, further including estimating blood pressure based, at least in part, on heart rate waveforms obtained by the sensor system.
11. A wearable device, including: a sensor system configured to obtain heart rate waveforms from a user of the wearable device, the sensor system being adjustable according to a plurality of wearable device configurations; a user interface system; and a control system configured to: obtain, via the sensor system, first heart rate waveforms at a first wearable device configuration, the first wearable device configuration corresponding with a first position of one or more sensors of the sensor system; control the user interface system to prompt the user to change the wearable device configuration to a second wearable device configuration, the second wearable device configuration corresponding with a second position of one or more sensors of the sensor system; obtain, via the sensor system, second heart rate waveforms at the second wearable device configuration; determine, based at least in part on the first heart rate waveforms and the second heart rate waveforms, whether to change a wearable device configuration or maintain a current wearable device configuration; and control the user interface system to prompt the user either to change the wearable device configuration or maintain the current wearable device configuration.
12. The wearable device of clause 11, where the determining further includes: determining a first signal-to-noise ratio (SNR) corresponding to the first heart rate waveforms; determining a second SNR corresponding to the second heart rate waveforms; and comparing the first SNR to the second SNR.
13. The wearable device of clause 11 or clause 12, where the control system is further configured to: obtain, via the sensor system, first through Nth heart rate waveforms at first through Nth wearable device configurations, N being an integer greater than 2; select, based at least in part on the first through Nth heart rate waveforms, one of the first through Nth wearable device configurations; and control the user interface system to prompt the user to change the wearable device configuration to a selected wearable device configuration.
14. The wearable device of any one of clauses 11-13, where the sensor system includes a photoacoustic sensor system and where the first heart rate waveforms and the second heart rate waveforms are obtained via a photoacoustic process.
15. The wearable device of clause 14, where photoacoustic sensor system includes a light source system and a receiver system, and where the photoacoustic process involves: controlling light source system to direct light to a target portion of a user's body; and obtaining, via the receiver system of the wearable device, acoustic signals corresponding to photoacoustic responses of the target portion of the user's body to the light.
16. The wearable device of any one of clauses 11-15, where the control system is further configured to: obtain sensor data at a current pressure being applied to a user's body by the wearable device at the current wearable device configuration; and determine, based at least in part on the sensor data, whether to prompt the user to change the current pressure to another pressure.
17. The wearable device of clause 16, where determining whether to prompt the user to change the current pressure to another pressure involves determining whether the current pressure is within a desired pressure range.
18. The wearable device of clause 16 or clause 17, where determining whether to prompt the user to change the current pressure to another pressure involves determining whether a blood vessel distension has been reduced at the current pressure.
19. The wearable device of any one of clauses 11-18, where the control system is further configured to control the user interface system to prompt the user to withdraw a hand used to change the wearable device configuration.
20. The wearable device of any one of clauses 11-19, where the control system is further configured to estimate blood pressure based, at least in part, on the heart rate waveforms obtained by the sensor system.
21. The wearable device of any one of clauses 11-20, where the control system is further configured to: determine a first signal-to-noise ratio (SNR) corresponding to the first heart rate waveforms; determine whether the first SNR equals or exceeds an SNR threshold; and responsive to determining that the first SNR equals or exceeds the SNR threshold, control the user interface system to prompt the user to maintain the current wearable device configuration.
22. A wearable device, including: a sensor system configured to obtain heart rate waveforms from a user of the wearable device, the sensor system being adjustable according to a plurality of wearable device configurations; a user interface system; and control means for: obtaining, via the sensor system, first heart rate waveforms at a first wearable device configuration, the first wearable device configuration corresponding with a first position of one or more sensors of the sensor system; controlling the user interface system to prompt the user to change the wearable device configuration to a second wearable device configuration, the second wearable device configuration corresponding with a second position of one or more sensors of the sensor system; obtaining, via the sensor system, second heart rate waveforms at the second wearable device configuration; determining, based at least in part on the first heart rate waveforms and the second heart rate waveforms, whether to change a wearable device configuration or maintain a current wearable device configuration; and controlling the user interface system to prompt the user either to change the wearable device configuration or maintain the current wearable device configuration.
23. The wearable device of clause 22, where the determining further includes: determining a first signal-to-noise ratio (SNR) corresponding to the first heart rate waveforms; determining a second SNR corresponding to the second heart rate waveforms; and comparing the first SNR to the second SNR.
24. The wearable device of clause 22 or clause 23, where the control means includes means for: obtaining, via the sensor system, first through Nth heart rate waveforms at first through Nth wearable device configurations, N being an integer greater than 2; selecting, based at least in part on the first through Nth heart rate waveforms, one of the first through Nth wearable device configurations; and controlling the user interface system to prompt the user to change the wearable device configuration to a selected wearable device configuration.
25. The wearable device of any one of clauses 22-24, where the sensor system includes a photoacoustic sensor system and where the first heart rate waveforms and the second heart rate waveforms are obtained via a photoacoustic process.
26. One or more computer-readable non-transitory media having instructions for controlling one or more devices to perform method stored thereon, the method including: obtaining, via a sensor system of a wearable device, first heart rate waveforms at a first wearable device configuration, the first wearable device configuration corresponding with a first position of one or more sensors of the sensor system; prompting, via a user interface of the wearable device, a user to change the wearable device configuration to a second wearable device configuration, the second wearable device configuration corresponding with a second position of one or more sensors of the sensor system; obtaining, via the sensor system, second heart rate waveforms at the second wearable device configuration; determining, based at least in part on the first heart rate waveforms and the second heart rate waveforms, whether to change a wearable device configuration or maintain a current wearable device configuration; and prompting, via the user interface, the user either to change the wearable device configuration or maintain the current wearable device configuration.
27. The one or more computer-readable non-transitory media of clause 26, where the determining further includes: determining a first signal-to-noise ratio (SNR) corresponding to the first heart rate waveforms; determining a second SNR corresponding to the second heart rate waveforms; and comparing the first SNR to the second SNR.
28. The one or more computer-readable non-transitory media of clause 26 or clause 27, where the method further includes: obtaining, via the sensor system, first through Nth heart rate waveforms at first through Nth wearable device configurations, N being an integer greater than 2; selecting, based at least in part on the first through Nth heart rate waveforms, one of the first through Nth wearable device configurations; and prompting, via the user interface, the user to change the wearable device configuration to a selected wearable device configuration.
29. The one or more computer-readable non-transitory media of any one of clauses 26-28, where the first heart rate waveforms and the second heart rate waveforms are obtained via a photoacoustic process.
30. The one or more computer-readable non-transitory media of clause 29, where the photoacoustic process involves: controlling a light source system of the wearable device to direct light to a target portion of a user's body; and obtaining, via a receiver system of the wearable device, acoustic signals corresponding to photoacoustic responses of the target portion of the user's body to the light.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

What is claimed is:

1. A method, comprising:
obtaining, via a sensor system of a wearable device, first heart rate waveforms at a first wearable device configuration, the first wearable device configuration corresponding with a first position of one or more sensors of the sensor system;
prompting, via a user interface of the wearable device, a user to change the wearable device configuration to a second wearable device configuration, the second wearable device configuration corresponding with a second position of one or more sensors of the sensor system;
obtaining, via the sensor system, second heart rate waveforms at the second wearable device configuration;
determining, based at least in part on the first heart rate waveforms and the second heart rate waveforms, whether to change a wearable device configuration or maintain a current wearable device configuration; and
prompting, via the user interface, the user either to change the wearable device configuration or maintain the current wearable device configuration.

2. The method of claim 1, wherein the determining further comprises:
determining a first signal-to-noise ratio (SNR) corresponding to the first heart rate waveforms;
determining a second SNR corresponding to the second heart rate waveforms; and
comparing the first SNR to the second SNR.

3. The method of claim 1, wherein the method further comprises:
obtaining, via the sensor system, first through N^{th} heart rate waveforms at first through N^{th} wearable device configurations, N being an integer greater than 2;
selecting, based at least in part on the first through N^{th} heart rate waveforms, one of the first through N^{th} wearable device configurations; and
prompting, via the user interface, the user to change the wearable device configuration to a selected wearable device configuration.

4. The method of claim 1, wherein the first heart rate waveforms and the second heart rate waveforms are obtained via a photoacoustic process.

5. The method of claim 4, wherein the photoacoustic process involves:
controlling a light source system of the wearable device to direct light to a target portion of a user's body; and
obtaining, via a receiver system of the wearable device, acoustic signals corresponding to photoacoustic responses of the target portion of the user's body to the light.

6. The method of claim 1, further comprising:
obtaining sensor data at a current pressure being applied to a user's body by the wearable device at the current wearable device configuration; and
determining, based at least in part on the sensor data, whether to prompt the user to change the current pressure to another pressure.

7. The method of claim 6, wherein determining whether to prompt the user to change the current pressure to another pressure involves determining whether the current pressure is within a desired pressure range.

8. The method of claim 6, wherein determining whether to prompt the user to change the current pressure to another pressure involves determining whether a blood vessel distension has been reduced at the current pressure.

9. The method of claim 1, further comprising prompting, via the user interface, the user to withdraw a hand used to change the wearable device configuration.

10. The method of claim 1, further comprising estimating blood pressure based, at least in part, on heart rate waveforms obtained by the sensor system.

11. A wearable device, comprising:
a sensor system configured to obtain heart rate waveforms from a user of the wearable device, the sensor system being adjustable according to a plurality of wearable device configurations;
a user interface system; and
a control system configured to:
obtain, via the sensor system, first heart rate waveforms at a first wearable device configuration, the first wearable device configuration corresponding with a first position of one or more sensors of the sensor system;
control the user interface system to prompt the user to change the wearable device configuration to a second wearable device configuration, the second wearable device configuration corresponding with a second position of one or more sensors of the sensor system;
obtain, via the sensor system, second heart rate waveforms at the second wearable device configuration;
determine, based at least in part on the first heart rate waveforms and the second heart rate waveforms, whether to change a wearable device configuration or maintain a current wearable device configuration; and
control the user interface system to prompt the user either to change the wearable device configuration or maintain the current wearable device configuration.

12. The wearable device of claim 11, wherein the determining further comprises:
determining a first signal-to-noise ratio (SNR) corresponding to the first heart rate waveforms;
determining a second SNR corresponding to the second heart rate waveforms; and
comparing the first SNR to the second SNR.

13. The wearable device of claim 11, wherein the control system is further configured to:
obtain, via the sensor system, first through N^{th} heart rate waveforms at first through N^{th} wearable device configurations, N being an integer greater than 2;
select, based at least in part on the first through N^{th} heart rate waveforms, one of the first through N^{th} wearable device configurations; and
control the user interface system to prompt the user to change the wearable device configuration to a selected wearable device configuration.

14. The wearable device of claim 11, wherein the sensor system includes a photoacoustic sensor system and wherein the first heart rate waveforms and the second heart rate waveforms are obtained via a photoacoustic process.

15. The wearable device of claim 14, wherein photoacoustic sensor system includes a light source system and a receiver system, and wherein the photoacoustic process involves:
controlling light source system to direct light to a target portion of a user's body; and
obtaining, via the receiver system of the wearable device, acoustic signals corresponding to photoacoustic responses of the target portion of the user's body to the light.

16. The wearable device of claim 11, wherein the control system is further configured to:
obtain sensor data at a current pressure being applied to a user's body by the wearable device at the current wearable device configuration; and
determine, based at least in part on the sensor data, whether to prompt the user to change the current pressure to another pressure.

17. The wearable device of claim 16, wherein determining whether to prompt the user to change the current pressure to another pressure involves determining whether the current pressure is within a desired pressure range.

18. The wearable device of claim 16, wherein determining whether to prompt the user to change the current pressure to another pressure involves determining whether a blood vessel distension has been reduced at the current pressure.

19. The wearable device of claim 11, wherein the control system is further configured to control the user interface system to prompt the user to withdraw a hand used to change the wearable device configuration.

20. The wearable device of claim 11, wherein the control system is further configured to estimate blood pressure based, at least in part, on the heart rate waveforms obtained by the sensor system.

21. The wearable device of claim 11, wherein the control system is further configured to:
determine a first signal-to-noise ratio (SNR) corresponding to the first heart rate waveforms;
determine whether the first SNR equals or exceeds an SNR threshold; and
responsive to determining that the first SNR equals or exceeds the SNR threshold, control the user interface system to prompt the user to maintain the current wearable device configuration.

22. A wearable device, comprising:
a sensor system configured to obtain heart rate waveforms from a user of the wearable device, the sensor system being adjustable according to a plurality of wearable device configurations;
a user interface system; and
control means for:
obtaining, via the sensor system, first heart rate waveforms at a first wearable device configuration, the first wearable device configuration corresponding with a first position of one or more sensors of the sensor system;
controlling the user interface system to prompt the user to change the wearable device configuration to a second wearable device configuration, the second wearable device configuration corresponding with a second position of one or more sensors of the sensor system;
obtaining, via the sensor system, second heart rate waveforms at the second wearable device configuration;
determining, based at least in part on the first heart rate waveforms and the second heart rate waveforms, whether to change a wearable device configuration or maintain a current wearable device configuration; and
controlling the user interface system to prompt the user either to change the wearable device configuration or maintain the current wearable device configuration.

23. The wearable device of claim 22, wherein the determining further comprises:
determining a first signal-to-noise ratio (SNR) corresponding to the first heart rate waveforms;
determining a second SNR corresponding to the second heart rate waveforms; and
comparing the first SNR to the second SNR.

24. The wearable device of claim 22, wherein the control means includes means for:
obtaining, via the sensor system, first through N^{th} heart rate waveforms at first through N^{th} wearable device configurations, N being an integer greater than 2;
selecting, based at least in part on the first through N^{th} heart rate waveforms, one of the first through N^{th} wearable device configurations; and
controlling the user interface system to prompt the user to change the wearable device configuration to a selected wearable device configuration.

25. The wearable device of claim 22, wherein the sensor system includes a photoacoustic sensor system and wherein the first heart rate waveforms and the second heart rate waveforms are obtained via a photoacoustic process.

26. One or more computer-readable non-transitory media having instructions for controlling one or more devices to perform method stored thereon, the method comprising:
obtaining, via a sensor system of a wearable device, first heart rate waveforms at a first wearable device configuration, the first wearable device configuration corresponding with a first position of one or more sensors of the sensor system;
prompting, via a user interface of the wearable device, a user to change the wearable device configuration to a second wearable device configuration, the second wearable device configuration corresponding with a second position of one or more sensors of the sensor system;
obtaining, via the sensor system, second heart rate waveforms at the second wearable device configuration;
determining, based at least in part on the first heart rate waveforms and the second heart rate waveforms, whether to change a wearable device configuration or maintain a current wearable device configuration; and
prompting, via the user interface, the user either to change the wearable device configuration or maintain the current wearable device configuration.

27. The one or more computer-readable non-transitory media of claim 26, wherein the determining further comprises:
determining a first signal-to-noise ratio (SNR) corresponding to the first heart rate waveforms;
determining a second SNR corresponding to the second heart rate waveforms; and
comparing the first SNR to the second SNR.

28. The one or more computer-readable non-transitory media of claim 26, wherein the method further comprises:
obtaining, via the sensor system, first through N^{th} heart rate waveforms at first through N^{th} wearable device configurations, N being an integer greater than 2;
selecting, based at least in part on the first through N^{th} heart rate waveforms, one of the first through N^{th} wearable device configurations; and
prompting, via the user interface, the user to change the wearable device configuration to a selected wearable device configuration.

29. The one or more computer-readable non-transitory media of claim 26, wherein the first heart rate waveforms and the second heart rate waveforms are obtained via a photoacoustic process.

30. The one or more computer-readable non-transitory media of claim 29, wherein the photoacoustic process involves:
controlling a light source system of the wearable device to direct light to a target portion of a user's body; and
obtaining, via a receiver system of the wearable device, acoustic signals corresponding to photoacoustic responses of the target portion of the user's body to the light.
